(19) United States
(12) Patent Application Publication
(10) Pub. No.: US 2017/0314074 A1
(43) Pub. Date: Nov. 2, 2017

(54) LPA-ASSOCIATED PROTEIN AND RNA EXPRESSION

(71) Applicant: Intermune, Inc., Brisbane, CA (US)

(72) Inventors: Karl Kossen, Burlingame, CA (US); Sharlene R. Lim, San Mateo, CA (US); Scott D. Seiwert, Half Moon Bay, CA (US); Donald Ruhrmund, San Francisco, CA (US)

(21) Appl. No.: 15/351,719

(22) Filed: Nov. 15, 2016

Related U.S. Application Data
(63) Continuation of application No. PCT/US2015/031427, filed on May 18, 2015.
(60) Provisional application No. 61/994,768, filed on May 16, 2014.

(51) Int. Cl. C12Q 1/68

(52) U.S. Cl. CPC C12Q 1/6883; C12Q 2600/118; C12Q 2600/158

(57) ABSTRACT

Provided herein are, inter alia, methods and biomarkers to determine if a patient has or is at risk for developing an LPA-associated disease, to indicate the progressiveness of the disease, and/or to facilitate evaluation of responsiveness to therapy. Compositions and kits including the biomarker proteins and biomarker RNAs provided herein are further provided.

### CROSS-REFERENCES TO RELATED APPLICATIONS

This application is a continuation of International Application No. PCT/US2015/031427, filed May 18, 2015, which claims priority to U.S. Provisional Application No. 61/994,768, filed May 16, 2014, the disclosures of which are incorporated herein in their entireties and for all purposes.

### BACKGROUND OF THE INVENTION

Lysophospholipids (such as lysophosphatidic acid (LPA)) affect important cellular functions including cellular proliferation, differentiation, survival, migration, adhesion, invasion, and morphogenesis. These functions impact many biological processes including neurogenesis, angiogenesis, wound healing, immunity, and carcinogenesis. LPA acts through specific G protein-coupled receptors (GPCRs) in an autocrine and paracrine fashion. LPA binds to its cognate GPCRs (LPA₁, LPA₂, LPA₃, LPA₄, LPA₅, and LPA₆) and thereby activates intracellular signaling pathways to produce a variety of biological responses. LPA is an important biological effector molecule with a diverse range of physiological actions (e.g., effects on blood pressure, platelet activation, smooth muscle contraction, cell growth, cell rounding, neurite retraction, actin stress fiber formation, cell migration). LPA effects are predominantly receptor mediated and activation of the LPA receptors (LPA₁, LPA₂, LPA₃, LPA₄, LPA₅, and LPA₆) with LPA mediates a range of downstream signaling cascades. Nearly all mammalian cells, tissues, and organs co-express several LPA-receptor subtypes indicating that LPA receptor signaling occurs in a cooperative manner.

There is a need in the art for well-defined biomarkers to determine if a patient has or is at risk for developing an Lysophosphatidic Acid (LPA)-associated disease, to indicate the progressiveness of the disease, and/or to facilitate evaluation of responsiveness to therapy. The invention provided herein addresses these and other needs in the art by providing, inter alia, novel protein biomarkers of LPA-associated disease.

### BRIEF SUMMARY OF THE INVENTION

In one aspect, a method of determining an expression level of an LPA-associated disease marker protein set forth in Table 1 or Table 2 in a subject that has or is at risk for developing an LPA-associated disease is provided. The method includes obtaining a biological sample from the subject and determining an expression level of an LPA-associated disease marker protein set forth in Table 1 or Table 2 in the biological sample.

In another aspect, a method of determining an expression level of an LPA-associated disease marker RNA set forth in Table 3 or Table 4 in a subject that has or is at risk for developing an LPA-associated disease is provided. The method includes obtaining a biological sample from the subject and determining an expression level of an LPA-associated disease marker RNA set forth in Table 3 or Table 4 in the biological sample.

In another aspect, a method of determining whether a subject has or is at risk of developing an LPA-associated disease is provided. The method includes (i) detecting an expression level of one or more LPA-associated disease marker proteins set forth in Table 1 or Table 2 in a subject. (ii) It is determined whether the expression level is increased or decreased relative to a standard control, wherein an elevated expression level of an LPA-associated disease marker protein set forth in Table 1 or Table 2 or a decreased expression level of an LPA-associated disease marker protein set forth in Table 1 or Table 2 relative to the standard control indicates that the subject has or is at risk of developing an LPA-associated disease. Based at least in part on the expression level in step (ii), it is determined whether the subject has or is at risk for developing an LPA-associated disease.

In another aspect, a method of determining whether a subject has or is at risk of developing an LPA-associated disease is provided. The method includes (i) detecting an expression level of one or more LPA-associated disease marker RNAs set forth in Table 3 or Table 4 in a subject. (ii) It is determined whether the expression level is increased or decreased relative to a standard control, wherein an elevated expression level of an LPA-associated disease marker RNA set forth in Table 3 or Table 4 or a decreased expression level of an LPA-associated disease marker RNA set forth in Table 3 or Table 4 relative to the standard control indicates that the subject has or is at risk of developing an LPA-associated disease. Based at least in part on the expression level in step (ii), it is determined whether the subject has or is at risk for developing an LPA-associated disease.

In another aspect, a method of determining an LPA-associated disease activity in a patient is provided. The method includes (i) determining a first expression level of a protein set forth in Table 1 or Table 2 in the patient at a first time point. (ii) A second expression level of a protein set forth in Table 1 or Table 2 in the patient is determined at a second time point. (iii) The second expression level of a protein set forth in Table 1 or Table 2 is compared to the first expression level of a protein set forth in Table 1 or Table 2, thereby determining the LPA-associated disease activity in the patient.

In another aspect, a method of determining an LPA-associated disease activity in a patient is provided. The method includes (i) determining a first expression level of an RNA set forth in Table 3 or Table 4 in the patient at a first time point. (ii) A second expression level of an RNA set forth in Table 3 or Table 4 is determined in the patient at a second time point. (iii) The second expression level of an RNA set forth in Table 3 or Table 4 is compared to the first expression level of an RNA set forth in Table 3 or Table 4, thereby determining the LPA-associated disease activity in the patient.

In another aspect, a method of treating an LPA-associated disease in a subject in need thereof is provided. The method includes administering to the subject an effective amount of an modulator of an LPA-associated disease marker protein set forth in Table 1 or Table 2, thereby treating an LPA-associated disease in the subject.

In another aspect, a method of treating an LPA-associated disease in a subject in need thereof is provided. The method includes administering to the subject an effective amount of an modulator of an LPA-associated disease marker RNA set forth in Table 3 or Table 4, thereby treating an LPA-associated disease in the subject.

In another aspect, a method of treating an LPA-associated disease in a subject in need thereof is provided. The method includes (i) determining whether a subject expresses an elevated level or a decreased level of an LPA-associated disease marker protein set forth in Table 1 or Table 2 relative to a standard control. (ii) When an elevated expression level or a decreased expression level of the LPA-associated disease marker protein set forth in Table 1 or Table 2 is found relative to the standard control, an LPA-associated disease treatment, an antagonist or an agonist of an LPA-associated disease marker protein set forth in Table 1 or Table 2 is administered to the subject, thereby treating the subject.

In another aspect, a method of treating an LPA-associated disease in a subject in need thereof is provided. The method includes (i) determining whether a subject expresses an elevated level or a decreased level of an LPA-associated disease marker RNA set forth in Table 3 or Table 4 relative to a standard control. (ii) When an elevated expression level or a decreased expression level of the LPA-associated disease marker RNA set forth in Table 3 or Table 4 is found relative to the standard control, an LPA-associated disease treatment, an antagonist or an agonist of an LPA-associated disease marker RNA set forth in Table 3 or Table 4 is administered to the subject, thereby treating the subject.

In another aspect, a method of determining whether an LPA-associated disease patient is at risk for progression of the LPA-associated disease is provided. The method includes (i) detecting an expression level of one or more LPA-associated disease marker proteins set forth in Table 1 or Table 2 in an LPA-associated disease patient. (ii) It is determined whether the expression level is modulated relative to a standard control, wherein a modulated expression level of an LPA-associated disease marker protein set forth in Table 1 or Table 2 relative to the standard control indicates that the LPA-associated disease patient is at risk for progression of the LPA-associated disease. (iii) Based at least in part on the expression level in step (ii), it is determined whether the LPA-associated disease patient is at risk for progression of the LPA-associated disease.

In another aspect, a method of determining whether an LPA-associated disease patient is at risk for progression of the LPA-associated disease is provided. The method includes (i) detecting an expression level of one or more LPA-associated disease marker RNAs set forth in Table 3 or Table 4 in an LPA-associated disease patient. (ii) It is determined whether the expression level is modulated relative to a standard control, wherein a modulated expression level of an LPA-associated disease marker RNA set forth in Table 3 or Table 4 relative to the standard control indicates that the LPA-associated disease patient is at risk for progression of the LPA-associated disease. (iii) Based at least in part on the expression level in step (ii), it is determined whether the LPA-associated disease patient is at risk for progression of the LPA-associated disease.

In another aspect, a method of determining whether an LPA-associated disease patient is at risk for progression of the LPA-associated disease is provided. The method includes (i) determining a first expression level of a protein set forth in Table 1 or Table 2 in the patient at a first time point. (ii) A second expression level of a protein set forth in Table 1 or Table 2 in the patient is determined at a second time point. (iii) The second expression level of a protein set forth in Table 1 or Table 2 is compared to the first expression level of a protein set forth in Table 1 or Table 2, wherein when the second expression level of a protein set forth in Table 1 or Table 2 is different from the first level of a protein set forth in Table 1 or Table 2, the patient is at risk for progression of the LPA-associated disease.

In another aspect, a method of determining an expression level of an LPA-associated disease marker protein of SEQ ID NO:1-202 in a subject that has or is at risk for developing an LPA-associated disease is provided. The method includes (i) obtaining a biological sample from the subject; and (ii) determining an expression level of an LPA-associated disease marker protein of SEQ ID NO:1-202 in the biological sample.

In another aspect, a method of determining an expression level of an LPA-associated disease marker RNA of SEQ ID NO:203-499 in a subject that has or is at risk for developing an LPA-associated disease is provided. The method includes (i) obtaining a biological sample from the subject; and (ii) determining an expression level of an LPA-associated disease marker RNA of SEQ ID NO:203-499 in the biological sample.

In another aspect, a method of determining whether a subject has or is at risk of developing an LPA-associated disease is provided. The method includes (i) detecting an expression level of one or more LPA-associated disease marker proteins of SEQ ID NO:1-202 in a subject. (ii) It is determined whether the expression level is increased or decreased relative to a standard control, wherein an elevated expression level of an LPA-associated disease marker protein of SEQ ID NO:1-202 or a decreased expression level of an LPA-associated disease marker protein of SEQ ID NO:1-202 relative to the standard control indicates that the subject has or is at risk of developing an LPA-associated disease. And (iii) based at least in part on the expression level in step (ii), determining whether the subject has or is at risk for developing an LPA-associated disease.

In another aspect, a method of determining whether a subject has or is at risk of developing an LPA-associated disease is provided. The method includes (i) detecting an expression level of one or more LPA-associated disease marker RNAs of SEQ ID NO:203-499 in a subject. (ii) It is determined whether the expression level is increased or decreased relative to a standard control, wherein an elevated expression level of an LPA-associated disease marker RNA of SEQ ID NO:203-499 or a decreased expression level of an LPA-associated disease marker RNA of SEQ ID NO:203-499 relative to the standard control indicates that the subject has or is at risk of developing an LPA-associated disease. And (iii) based at least in part on the expression level in step (ii), determining whether the subject has or is at risk for developing an LPA-associated disease.

In another aspect, a method of determining an LPA-associated disease activity in a patient is provided. The method includes (i) determining a first expression level of a protein of SEQ ID NO:1-202 in the patient at a first time point. (ii) A second expression level of a protein of SEQ ID NO:1-202 in the patient is determined at a second time point. (iii) The second expression level of a protein of SEQ ID NO:1-202 is compared to the first expression level of a protein of SEQ ID NO:1-202, thereby determining the LPA-associated disease activity in the patient.

In another aspect, a method of determining an LPA-associated disease activity in a patient is provided. The method includes (i) determining a first expression level of an RNA of SEQ ID NO:203-499 in the patient at a first time point. (ii) A second expression level of an RNA of SEQ ID NO:203-499 is determined in the patient at a second time point. (iii) The second expression level of an RNA of SEQ ID NO:203-499 is compared to the first expression level of an RNA of SEQ ID NO:203-499, thereby determining the LPA-associated disease activity in the patient.

In another aspect, a method of treating an LPA-associated disease in a subject in need thereof is provided. The method includes administering to the subject an effective amount of an modulator of an LPA-associated disease marker protein of SEQ ID NO:1-202, thereby treating an LPA-associated disease in the subject.

In another aspect, a method of treating an LPA-associated disease in a subject in need thereof is provided. The method includes administering to the subject an effective amount of an modulator of an LPA-associated disease marker RNA of SEQ ID NO:203-499, thereby treating an LPA-associated disease in the subject.

In another aspect, a method of treating an LPA-associated disease in a subject in need thereof is provided. The method includes (i) determining whether a subject expresses an elevated level or a decreased level of an LPA-associated disease marker protein of SEQ ID NO:1-202 relative to a standard control. And (ii) when an elevated expression level or a decreased expression level of the LPA-associated disease marker protein of SEQ ID NO:1-202 is found relative to the standard control, administering to the subject an LPA-associated disease treatment, an antagonist or an agonist of an LPA-associated disease marker protein of SEQ ID NO:1-202, thereby treating the subject.

In another aspect, a method of treating an LPA-associated disease in a subject in need thereof is provided. The method includes (i) determining whether a subject expresses an elevated level or a decreased level of an LPA-associated disease marker RNA of SEQ ID NO:203-499 relative to a standard control. And (ii) when an elevated expression level or a decreased expression level of the LPA-associated disease marker RNA of SEQ ID NO:203-499 is found relative to the standard control, administering to the subject an LPA-associated disease treatment, an antagonist or an agonist of an LPA-associated disease marker RNA of SEQ ID NO:203-499, thereby treating the subject.

In another aspect, a method of determining whether an LPA-associated disease patient is at risk for progression of the LPA-associated disease is provided. The method includes (i) detecting an expression level of one or more LPA-associated disease marker proteins set forth of SEQ ID NO:1-202 in an LPA-associated disease patient. (ii) It is determined whether the expression level is modulated relative to a standard control, wherein a modulated expression level of an LPA-associated disease marker protein of SEQ ID NO:1-202 relative to the standard control indicates that the LPA-associated disease patient is at risk for progression of the LPA-associated disease; and (iii) based at least in part on the expression level in step (ii), determining whether the LPA-associated disease patient is at risk for progression of the LPA-associated disease.

In another aspect, a method of determining whether an LPA-associated disease patient is at risk for progression of the LPA-associated disease is provided. The method includes (i) detecting an expression level of one or more LPA-associated disease marker RNAs of SEQ ID NO:203-499 in an LPA-associated disease patient. (ii) It is determined whether said expression level is modulated relative to a standard control, wherein a modulated expression level of an LPA-associated disease marker RNA of SEQ ID NO:203-499 relative to the standard control indicates that the LPA-associated disease patient is at risk for progression of said LPA-associated disease; and (iii) based at least in part on the expression level in step (ii), determining whether the LPA-associated disease patient is at risk for progression of the LPA-associated disease.

In another aspect, a complex in vitro is provided. The complex includes a marker protein binding agent bound to a LPA-associated disease marker protein of SEQ ID NO:1-202 or fragment thereof, wherein the LPA-associated disease marker protein is extracted from a human subject having or at risk of developing an LPA-associated disease.

In another aspect, a complex in vitro is provided. The complex includes a marker RNA binding agent bound to a LPA-associated disease marker RNA of SEQ ID NO:203-499 or fragment thereof, wherein the LPA-associated disease marker RNA is extracted from a human subject having or at risk of developing an LPA-associated disease.

In another aspect, a kit is provided. The kit includes (a) a marker protein binding agent capable of binding to a substance within a biological sample from a human subject having or at risk of developing an LPA-associated disease; wherein the substance is an LPA-associated disease marker protein of SEQ ID NO:1-202 or fragment thereof. And (b) a detecting reagent or a detecting apparatus capable of indicating binding of the marker protein binding agent to the substance.

In another aspect, a kit is provided. The kit includes (a) a marker RNA binding agent capable of binding to a substance within a biological sample from a human subject having or at risk of developing an LPA-associated disease; wherein the substance is an LPA-associated disease marker RNA of SEQ ID NO:203-499 or fragment thereof. And (b) a detecting reagent or a detecting apparatus capable of indicating binding of the marker RNA binding agent to the substance.

### BRIEF DESCRIPTION OF THE DRAWINGS

FIG. 1: LPA receptor expression in cells. Expression of LPA receptors 1-6 was assayed by qRT-PCR in primary human fibroblasts, endothelial cells, epithelial cells, and pericytes.

FIG. 2: Experimental Design for Gene Expression and Secreted Protein Analysis.

FIG. 3: LPA-Induced mRNAs in Primary Pulmonary Fibroblasts.

FIG. 4: Comparison of 1 μM LPA induction in Fibroblasts and Epithelial Cells.

FIG. 5: Selected Differentially Secreted Proteins from Fibroblasts.

FIG. 6: LPA-Induced Proteins Independently Associated With IPF.

### DETAILED DESCRIPTION OF THE INVENTION

### I. Definitions

The term “disease” refers to any deviation from the normal health of a mammal and includes a state when disease symptoms are present, as well as conditions in which a deviation (e.g., infection, gene mutation, genetic defect, etc.) has occurred, but symptoms are not yet manifested. According to the present invention, the methods disclosed herein are suitable for use in a patient that is a member of the Vertebrate class, Mammalia, including, without limitation, primates, livestock and domestic pets (e.g., a companion animal). Typically, a patient will be a human patient.

The terms “pulmonary disease,” “pulmonary disorder,” “lung disease,” etc. are used interchangeably herein. The term is used to broadly refer to lung disorders characterized by difficulty breathing, coughing, airway discomfort and inflammation, increased mucus, and/or pulmonary fibrosis.

The term “LPA-associated disease” is used to broadly refer to disorders or symptoms of disease associated with LPA function. In embodiments, the disease is caused by, or a symptom of the disease is caused by aberrant LPA function. LPA function as described herein refers to any cellular function affected by LPA and includes without limitation cellular proliferation, differentiation, survival, migration, adhesion, invasion, morphogenesis, neurogenesis, angiogenesis, wound healing, immunity, and carcinogenesis.

The term “associated” or “associated with” in the context of a substance (e.g., LPA) or substance activity (e.g., LPA activity) or substance function (e.g., LPA function) associated with a disease means that the disease is caused by (in whole or in part), or a symptom of the disease is caused by (in whole or in part) the substance or substance activity or function (i.e., LPA, LPA activity, LPA function). For example, a disease associated with LPA or a symptom of an LPA-associated disease or condition associated with an increase or decrease in LPA activity may be a disease or symptom that results (entirely or partially) from an increase or decrease in LPA activity (e.g. increase or decrease in LPA activity or increase or decrease in activity of an LPA signal transduction or signaling pathway). The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

Non-limiting examples of LPA-associated diseases are idiopathic pulmonary fibrosis, pulmonary fibrosis, bronchiolitis obliterans, chronic lung transplant rejection, scleroderma, primary focal segmental glomerulosclerosis (FSGC) or membranoproliferative glomerulonephritis (MPGN), idiopathic interstitial pneumonia, interstitial lung disease in systemic sclerosis, a fibrosis condition of the lung, autoimmune lung diseases, benign prostate hypertrophy, coronary or myocardial infarction, atrial fibrillation, cerebral infarction, myocardial fibrosis, musculoskeletal fibrosis, post-surgical adhesions, liver cirrhosis, renal fibrotic disease, fibrotic vascular disease, scleroderma, Hermansky-Pudlak syndrome, neurofibromatosis, Alzheimer's disease, diabetic retinopathy, or skin lesions, lymph node fibrosis associated with HIV, chronic obstructive pulmonary disease (COPD), inflammatory pulmonary fibrosis, rheumatoid arthritis; rheumatoid spondylitis; osteoarthritis; gout, other arthritic conditions; sepsis; septic shock; endotoxic shock; gram-negative sepsis; toxic shock syndrome; myofacial pain syndrome (MPS); Shigellosis; asthma; adult respiratory distress syndrome; inflammatory bowel disease; Crohn's disease; psoriasis; eczema; ulcerative colitis; glomerular nephritis; scleroderma; chronic thyroiditis; Grave's disease; Ormond's disease; autoimmune gastritis; myasthenia gravis; autoimmune hemolytic anemia; autoimmune neutropenia; thrombocytopenia; pancreatic fibrosis; chronic active hepatitis including hepatic fibrosis; acute or chronic renal disease; renal fibrosis; diabetic nephropathy; irritable bowel syndrome; pyresis; restenosis; cerebral malaria; stroke or ischemic injury; neural trauma; Alzheimer's disease; Huntington's disease; Parkinson's disease; acute or chronic pain; allergies, including allergic rhinitis or allergic conjunctivitis; cardiac hypertrophy, chronic heart failure; acute coronary syndrome; cachexia; malaria; leprosy; leishmaniasis; Lyme disease; Reiter's syndrome; acute synoviitis; muscle degeneration, bursitis; tendonitis; tenosynoviitis; herniated, ruptured, or prolapsed intervertebral disk syndrome; osteopetrosis; thrombosis; silicosis; pulmonary sarcosis; bone resorption diseases, such as osteoporosis or multiple myeloma-related bone disorders; cancer, including but not limited to metastatic breast carcinoma, colorectal carcinoma, malignant melanoma, gastric cancer, or non-small cell lung cancer; graft-versus-host reaction; or auto-immune diseases, such as multiple sclerosis, lupus or fibromyalgia; AIDS or other viral diseases such as Herpes Zoster, Herpes Simplex I or II, influenza virus, Severe Acute Respiratory Syndrome (SARS) or cytomegalovirus; or diabetes mellitus, proliferative disorders (including both benign or malignant hyperplasia), acute myelogenous leukemia, chronic myelogenous leukemia, Kaposi's sarcoma, metastatic melanoma, multiple myeloma, breast cancer, including metastatic breast carcinoma; colorectal. carcinoma; malignant melanoma; gastric cancer; non-small cell lung cancer (NSCLC); bone metastases; pain disorders including neuromuscular pain, headache, cancer pain, dental pain, or arthritis pain; angiogenic disorders including solid tumor angiogenesis, ocular neovascularization, or infantile hemangioma; conditions associated with the cyclooxygenase or lipoxygenase signaling pathways, including conditions associated with prostaglandin endoperoxide synthase-2 (including edema, fever, analgesia, or pain); organ hypoxia; thrombin-induced platelet aggregation; or protozoal diseases. For example, IPF and scleroderma (or systemic sclerosis) associated interstitial lung disease (SSc-ILD) share overlapping pathologic pathways, most notably the activation and proliferation of fibroblasts, expression of fibrogenic cytokines and growth factors, and progressive interstitial fibrosis.

The terms “subject,” “patient,” “individual,” and the like as used herein are not intended to be limiting and can be generally interchanged. That is, an individual described as a “patient” does not necessarily have a given disease, but may be merely seeking medical advice.

The term “subject” as used herein includes all members of the animal kingdom prone to suffering from the indicated disorder. In some aspects, the subject is a mammal, and in some aspects, the subject is a human.

A “control” sample or value refers to a sample that serves as a reference, usually a known reference, for comparison to a test sample. For example, a test sample can be taken from a patient suspected of having an LPA-associated disease and compared to samples from a known LPA-associated disease patient, or a known normal (non-disease) individual. A control can also represent an average value gathered from a population of similar individuals, e.g., LPA-associated disease patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein, the terms “pharmaceutically” acceptable is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose will generally refer to the amount of LPA-associated disease treatment, agonist or antagonist. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical, and depends on the route of administration. For example, a dosage form can be in a liquid form for nebulization, e.g., for inhalants, in a tablet or liquid, e.g., for oral delivery, or a saline solution, e.g., for injection.

As used herein, the terms “treat” and “prevent” are not intended to be absolute terms. Treatment can refer to any delay in onset, reduction in the frequency or severity of symptoms, amelioration of symptoms, improvement in patient comfort and/or respiratory function, etc. The effect of treatment can be compared to an individual or pool of individuals not receiving a given treatment, or to the same patient prior to, or after cessation of, treatment.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of LPA-associated disease symptoms in a patient. As indicated above, the prevention may be complete (no detectable symptoms) or partial, such that fewer symptoms are observed than would likely occur absent treatment.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “diagnosis” refers to a relative probability that an LPA-associated disease is present in the subject. Similarly, the term “prognosis” refers to a relative probability that a certain future outcome may occur in the subject. For example, in the context of the present invention, prognosis can refer to the likelihood that an individual will develop an LPA-associated disease, or the likely severity of the disease (e.g., severity of symptoms, rate of functional decline, survival, etc.). The terms are not intended to be absolute, as will be appreciated by any one of skill in the field of medical diagnostics.

The terms “correlating” and “associated,” in reference to determination of a LPA-associated disease risk factor, refers to comparing the presence or amount of the risk factor (e.g., decreased or increased expression of an LPA-associated biomarker protein) in an individual to its presence or amount in persons known to suffer from, or known to be at risk of, the LPA-associated disease, or in persons known to be free of LPA-associated disease, and assigning an increased or decreased probability of having/developing the LPA-associated disease to an individual based on the assay result(s).

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids, including ribonucleic acids (RNA) and deoxyribonucleic acids (DNA), and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

The terms “identical” or percent “identity,” in the context of two or more nucleic acids (e.g., genomic sequences or subsequences or coding sequences) or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%, or more identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 10 to about 100, about 20 to about 75, about 30 to about 50 amino acids or nucleotides in length.

An example of algorithms suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. As will be appreciated by one of skill in the art, the software for performing BLAST analyses is publicly available through the website of the National Center for Biotechnology Information (ncbi.nlm.nih.gov).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

“Antibody” refers to a polypeptide comprising a framework region from an immunoglobulin gene or fragments thereof that specifically binds and recognizes an antigen, e.g., a specific bacterial antigen. Typically, the “variable region” contains the antigen-binding region of the antibody (or its functional equivalent) and is most critical in specificity and affinity of binding. See Paul, Fundamental Immunology (2003).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term “aptamer” as provided herein refers to short oligonucleotides (e.g. deoxyribonucleotides), which fold into diverse and intricate molecular structures that bind with high affinity and specificity to proteins, peptides, and small molecules in a non-Watson Crick manner. An aptamer can thus be used to detect or otherwise target nearly any molecule of interest, including an LPA-associated disease marker protein. Methods of constructing and determining the binding characteristics of aptamers are well known in the art. For example, such techniques are described in U.S. Pat. Nos. 5,582,981, 5,595,877 and 5,637,459. Aptamers are typically at least 5 nucleotides, 10, 20, 30 or 40 nucleotides in length, and can be composed of modified nucleic acids to improve stability. Flanking sequences can be added for structural stability, e.g., to form 3-dimensional structures in the aptamer. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamers' chemical diversity. As a result high affinity aptamers for almost any protein target are enriched and identified.

A “biomarker,” “marker protein, “biomarker protein,” “marker RNA, or “biomarker RNA” as provided herein refers to any assayable characteristics or compositions that are used to identify, predict, or monitor a condition (e.g., an LPA-associated disease or lack thereof) or a therapy for said condition in a subject or sample. A biomarker is, for example, a protein or combination of proteins, an RNA or a combination of RNAs whose presence, absence, or relative amount is used to identify a condition (e.g. an LPA-associated disease) or status of a condition (e.g. an LPA-associated disease) in a subject or sample. Biomarkers identified herein are measured to determine levels, expression, activity, or to detect fragments, variants or homologs of said biomarkers. Variants include amino acid or nucleic acid variants or post translationally modified variants. In embodiments, the marker protein is a protein or fragment thereof as set forth in Table 1, Table 2 or Table 5. In embodiments, the marker protein is a homolog of the protein listed in Table 1, Table 2 or Table 5. In embodiments, the marker protein is a protein of SEQ ID NO:1-202 or fragment thereof. In embodiments, the marker protein is a homolog of the protein of SEQ ID NO:1-202. The marker proteins provided herein are identified by accession numbers referring to the corresponding amino acid and/or nucleic acid sequence of the individual marker proteins. Therefore, a person of ordinary skill in the art will immediately recognize the sequences of the marker proteins provided herein.

In embodiments, the marker RNA is an RNA of SEQ ID NO:203-499 or fragment thereof. In embodiments, the marker RNA is a homolog of the RNA of SEQ ID NO:203-499. In embodiments, the marker RNA complementary to SEQ ID NO:203-499 or fragments thereof. In embodiments, the marker RNA includes a sequence of at least 20 nucleotides complementary to SEQ ID NO:203-499 or fragments thereof. In embodiments, the marker RNA includes a sequence of at least 30 nucleotides complementary to SEQ ID NO:203-499 or fragments thereof. In embodiments, the marker RNA includes a sequence of at least 40 nucleotides complementary to SEQ ID NO:203-499 or fragments thereof. In embodiments, the marker RNA includes a sequence of at least 50 nucleotides complementary to SEQ ID NO:203-499 or fragments thereof In embodiments, the marker RNA includes a sequence of at least 60 nucleotides complementary to SEQ ID NO:203-499 or fragments thereof. In embodiments, the marker RNA includes a sequence of at least 70 nucleotides complementary to SEQ ID NO:203-499 or fragments thereof. In embodiments, the marker RNA includes a sequence of at least 80 nucleotides complementary to SEQ ID NO:203-499 or fragments thereof. In embodiments, the marker RNA includes a sequence of at least 90 nucleotides complementary to SEQ ID NO:203-499 or fragments thereof. In embodiments, the marker RNA includes a sequence of at least 100 nucleotides complementary to SEQ ID NO:203-499 or fragments thereof. In embodiments, the marker RNA includes about 50 nucleotides complementary to SEQ ID NO:203-499 or fragments thereof. In embodiments, the marker RNA is sequence at least 50%, 55%, 60%, 65,%, 70%, 75%, 80%, 85%, 90%, 95%, 96%, 97%, 98%, 99% or 100% identical to SEQ ID NO:203-499. In embodiments, the marker RNA has a sequence at least 50%, 55%, 60%, 65,%, 70%, 75%, 80%, 85%, 90%, 95%, 96%, 97%, 98%, 99% or 100% identical to the complement of SEQ ID NO:203-499.

In embodiments, the marker RNA is an RNA or fragment thereof as set forth in Table 3 or Table 4. In embodiments, the marker RNA is a homolog of the RNA listed in Table 3 or Table 4. The marker RNAs provided herein are identified by accession numbers referring to the corresponding nucleic acid sequence of the individual marker RNAs. Therefore, a person of ordinary skill in the art will immediately recognize the sequences of the marker RNAs provided herein.

In some examples of the disclosed methods, when the expression level of a biomarker(s) is assessed, the level is compared with control expression level of the biomarker(s). By control level is meant the expression level of a particular biomarker(s) from a sample or subject lacking a disease (e.g. an LPA-associated disease), at a selected stage of a disease or disease state, or in the absence of a particular variable such as a therapeutic agent. Alternatively, the control level comprises a known amount of biomarker. Such a known amount correlates with an average level of subjects lacking a disease, at a selected stage of a disease or disease state, or in the absence of a particular variable such as a therapeutic agent. A control level also includes the expression level of one or more biomarkers from one or more selected samples or subjects as described herein. For example, a control level includes an assessment of the expression level of one or more biomarkers in a sample from a subject that does not have a disease (e.g. an LPA-associated disease), is at a selected stage of progression of a disease (e.g. an LPA-associated disease), or has not received treatment for a disease. Another exemplary control level includes an assessment of the expression level of one or more biomarkers in samples taken from multiple subjects that do not have a disease, are at a selected stage of progression of a disease, or have not received treatment for a disease.

When the control level includes the expression level of one or more biomarkers in a sample or subject in the absence of a therapeutic agent, the control sample or subject is optionally the same sample or subject to be tested before or after treatment with a therapeutic agent or is a selected sample or subject in the absence of the therapeutic agent. Alternatively, a control level is an average expression level calculated from a number of subjects without a particular disease. A control level also includes a known control level or value known in the art.

In one particular example, a biomarker is a protein or combination of proteins whose expression level in a subject or sample is indicative of an LPA-associated disease or an LPA-associated disease activity. The expression level of a biomarker or a combination of a plurality of biomarkers may be increased or decreased compared to a control level. For example, the expression level of a biomarker or a combination of a plurality of biomarkers as provided herein may be increased or decreased in a subject compared to the expression level of the same subject at an earlier time point. Therefore, the expression level of a biomarker as provided herein may be indicative of a specific disease stage. Alternatively, the biomarker may be indicative of the efficacy of treatment. In other words, the expression level of a biomarker may be indicative of whether a patient is responsive to a treatment. The biomarker may further be indicative of the activity of a disease, wherein the activity of a disease refers to the change of one or more biomarker expression levels over the course of the disease.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

### II. Methods

Provided herein, inter alia, are biomarkers (e.g. protein biomarkers, RNA biomarkers) for diagnosing LPA-associated diseases as well as evaluation of progression, activity and treatment of LPA-associated diseases. Further provided herein are therapeutic targets for ameliorating LPA-associated diseases and compositions including an LPA-associated biomarker bound to a marker protein binding agent (e.g., an antibody).

In one aspect, a method of determining an expression level of an LPA-associated disease marker protein set forth in Table 1 or Table 2 in a subject that has or is at risk for developing an LPA-associated disease is provided. The method includes obtaining a biological sample from the subject and determining an expression level of an LPA-associated disease marker protein set forth in Table 1 or Table 2 in the biological sample.

In another aspect, a method of determining an expression level of an LPA-associated disease marker protein of SEQ ID NO:1-202 in a subject that has or is at risk for developing an LPA-associated disease is provided. The method includes (i) obtaining a biological sample from the subject; and (ii) determining an expression level of an LPA-associated disease marker protein of SEQ ID NO:1-202 in the biological sample.

In embodiments, the determining includes (a) contacting the LPA-associated disease marker protein with a marker protein binding agent in the biological sample, thereby forming a disease marker protein-binding agent complex; and (b) detecting the disease marker protein-binding agent complex. In embodiments, the marker protein binding agent includes a detectable moiety. In embodiments, the marker protein binding agent includes a capturing moiety. In embodiments, the capturing moiety is a cleavable capturing moiety. In embodiments, the detecting includes contacting the disease marker protein-binding agent complex with a capturing agent, thereby forming a captured disease marker protein-binding agent complex. In embodiments, the detecting further includes (1) contacting the captured disease marker protein-binding agent complex with a tagging moiety; thereby forming a tagged disease marker protein-binding agent complex; and (2) separating the tagged disease marker protein-binding agent complex from the biological sample. In embodiments, the detecting further includes after the separating of step (2) separating the capturing binding agent from the tagged disease marker protein-binding agent complex, thereby forming a cleaved disease marker protein-binding agent complex. In embodiments, the detecting further includes (3) separating the marker protein binding agent from the cleaved disease marker protein-binding agent complex; thereby forming a released marker protein binding agent; and (4) determining an amount of the released marker protein binding agent.

In embodiments, the method includes selecting a subject that has or is at risk for developing an LPA-associated disease. The selected subject may be treated for an LPA-associated disease. In some embodiments, the subject is not treated for an LPA-associated disease. The subject may be part of a plurality of subjects participating in a clinical trial. Wherein the subject is part of a clinical trial, the selecting is at least part based on the determining of an expression level as provided herein.

An LPA-associated disease marker protein is a biomarker protein useful to identify, predict, or monitor an LPA-associated disease or lack thereof or a therapy for an LPA-associated disease in a subject or sample. A person of ordinary skill in the art will immediately recognize that determining an expression level of an LPA-associated disease marker protein or an LPA-associated marker RNA described herein includes determining the level of one or more LPA-associated disease marker proteins or LPA-associated disease marker RNAs in a sample (e.g. patient biological sample such as a blood-derived biological sample). Thus is some embodiments, the expression level of a plurality of LPA-associated disease marker proteins or LPA-associated disease marker RNAs is determined. Wherein the expression level of a plurality of LPA-associated disease marker proteins or LPA-associated disease marker RNAs is determined, the level of at least two (e.g. 3, 4, 5, 6, 7, 8, 9, 10 etc.) LPA-associated disease marker proteins or LPA-associated disease marker RNAs is determined and the at least two LPA-associated disease marker proteins or LPA-associated disease marker RNAs are independently different.

In some embodiments, the LPA-associated disease is a fibrotic pulmonary disease. In embodiments, the fibrotic pulmonary disease is idiopathic pulmonary fibrosis or familial interstitial pneumonia. In embodiments, the fibrotic pulmonary disease is a progressive form of idiopathic pulmonary fibrosis. In other embodiments, the LPA-associated disease marker protein is a progressive fibrotic pulmonary disease marker protein or a progressive fibrotic pulmonary disease marker RNA. A progressive fibrotic pulmonary disease maker protein or RNA is a biomarker protein or RNA indicative of a fibrotic pulmonary disease patient having or being at risk of developing progressive fibrotic pulmonary disease (e.g., a progressive form of a fibrotic pulmonary disease). Thus, in some embodiments, the subject has or is at risk for developing a progressive fibrotic pulmonary disease. In other embodiments, the progressive fibrotic pulmonary disease is idiopathic pulmonary fibrosis. A progressive fibrotic pulmonary disease is a disease wherein certain clinical or physiological parameters decline over the course of the disease. Commonly used parameters to determine fibrotic pulmonary disease progression include for example, breathing metrics, such as forced expiratory volume (FEV1), vital capacity (VC), forced vital capacity (FVC), FEV1/FVC and diffusing capacity of carbon monoxide (DL_{CO}). For example, a fibrotic pulmonary disease patient showing a 5% decline in FVC as compared to a control level may be considered a progressive fibrotic pulmonary disease patient. A control level may be the FVC of the same patient measured at an earlier stage of the fibrotic pulmonary disease or the FVC calculated from a number of subjects lacking the fibrotic pulmonary disease. Thus, in some embodiments, the FVC of a progressive fibrotic pulmonary disease patient is at least 5% less than a control level.

In embodiments, the biological sample is a blood-derived biological sample, a urine-derived biological sample or a saliva-derived biological sample of the subject. In embodiments, the blood-derived biological sample is whole blood, serum or plasma.

The methods provided herein including embodiments thereof further include treating a subject for LPA-associated diseases. As described above the expression level of one or more LPA-associated disease marker proteins or LPA-associated disease marker RNAs is determined. In embodiments, the expression level of one or more LPA-associated disease marker RNAs is determined. In embodiments, an expression level of an LPA-associated disease marker protein of SEQ ID NO:1-202 is determined. In embodiments, an expression level of an LPA-associated disease marker RNA of SEQ ID NO:203-499 is determined.

The expression levels of the one or more LPA-associated disease marker proteins of SEQ ID NO:1-202 may be increased or decreased in an LPA-associated disease patient. The expression levels of the one or more LPA-associated disease marker RNAs of SEQ ID NO:203-499 may be increased or decreased in an LPA-associated disease patient. In some embodiments, the one or more LPA-associated disease marker proteins of SEQ ID NO:1-202 is increased. In some embodiments, the one or more LPA-associated disease marker proteins of SEQ ID NO:1-202 is decreased. In some embodiments, the one or more LPA-associated disease marker RNAs of SEQ ID NO:203-499 is increased. In some embodiments, the one or more LPA-associated disease marker RNAs of SEQ ID NO:203-499 is decreased. In embodiments, the expression level of the LPA-associated disease marker protein of SEQ ID NO:1-202 is elevated relative to a standard control. In embodiments, the expression level of the LPA-associated disease marker protein of SEQ ID NO:1-202 is deceased relative to a standard control. In embodiments, the expression level of the LPA-associated disease marker RNA of SEQ ID NO:203-499 is elevated relative to a standard control. In embodiments, the expression level of the LPA-associated disease marker RNA of SEQ ID NO:203-499 is deceased relative to a standard control.

A treatment regimen for an LPA-associated disease patient with modulated expression levels of one or more LPA-associated disease marker proteins or LPA-associated disease marker RNAs as disclosed herein (e.g. in Table 1, 2, 3, 4 or 5) may be administering to the patient an effective amount of a modulator affecting the one or more increased or decreased biomarker protein or biomarker RNA expression levels. Therefore, in some embodiments, the method includes administering to the subject an effective amount of a modulator of a modulator of the LPA-associated disease marker protein set forth in Table 1 or Table 2. In embodiments, the method includes administering to the subject an effective amount of a modulator of a modulator of the LPA-associated disease marker protein of SEQ ID NO:1-202. In embodiments, the modulator is an antagonist. In embodiments, the antagonist is a peptide, small molecule, nucleic acid, antibody or aptamer. In embodiments, the expression level of the LPA-associated disease marker protein set forth in Table 1 or Table 2 is elevated relative to a standard control. In embodiments, the expression level of the LPA-associated disease marker protein of SEQ ID NO:1-202 is elevated relative to a standard control. In embodiments, the modulator is an agonist. In embodiments, the agonist is a peptide, small molecule, nucleic acid, antibody or aptamer. In embodiments, the expression level of the LPA-associated disease marker protein of SEQ ID NO:1-202 is decreased relative to a standard control. In embodiments, the expression level of the LPA-associated disease marker protein set forth in Table 1 or Table 2 is decreased relative to a standard control. In embodiments, the method includes administering to the subject an effective amount of a further therapeutic agent.

The methods provided herein may include combinatorial treatment of a LPA-associated disease patient with a modulator of one or more LPA-associated disease marker proteins and a fibrotic pulmonary disease treatment. Thus, the method includes administering to the subject an effective amount of a further therapeutic agent. Exemplary therapeutic agents include, but are not limited to, the agents selected from the group consisting of steroids (including but not limited to prednisolone), cytotoxic agents (including but not limited to azathioprine and cyclophosphamide), bardoxolone, LPA agonists (including but not limited to AM152); Torisel (temsirolimus); PI3K inhibitors; pentraxin or serum amyloid P (including but not limited to Pentraxin-2 (PTX-2 or PRM-151)); MEK inhibitors (including but not limited to ARRY-162 and ARRY-300); p38 inhibitors; PAI-1 inhibitors (including but not limited to Tiplaxtinin); agents that reduce the activity of transforming growth factor-beta (TGF-β) (including but not limited to GC-1008 (Genzyme/Medlmmune); lerdelimumab (CAT-152; Trabio, Cambridge Antibody); metelimumab (CAT-192, Cambridge Antibody); LY-2157299 (Eli Lilly); ACU-HTR-028 (Opko Health)) including antibodies that target one or more TGF-β isoforms, inhibitors of TGF-β receptor kinases TGFBR1 (ALK5) and TGFBR2, and modulators of post-receptor signaling pathways; chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610; PS-433540; S-0139; sitaxentan sodium; TBC-3711; zibotentan); agents that reduce the activity of connective tissue growth factor (CTGF) (including but not limited to FG-3019, FibroGen), and including other CTGF-neutralizing antibodies; matrix metalloproteinase (MMP) inhibitors (including but not limited to MMPI-12, PUP-1 and tigapotide triflutate); agents that reduce the activity of epidermal growth factor receptor (EGFR) including but not limed to erlotinib, gefitinib, BMS-690514, cetuximab, antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Rα) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573 or rapamycin); agents that reduce levels of copper such as tetrathiomolybdate; agents that reduce oxidative stress including N-acetyl cysteine and tetrathiomolybdate; and interferon gamma, inhibitors of phosphodiesterase 4 (PDE4) (including but not limited to Roflumilast); inhibitors of phosphodiesterase 5 (PDES) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton), compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil, P-1894B, and safironil) and peroxisome proliferator-activated receptor (PPAR)-gamma agonists (including but not limited to pioglitazone and rosiglitazone), and combinations thereof. In some embodiments, the therapeutic agent is an anti-fibrotic drug. In some embodiments, the therapeutic agent is an idiopathic pulmonary fibrosis drug. In other embodiment, the idiopathic pulmonary fibrosis drug is a mucolytic drug.

In embodiments, the LPA-associated disease marker protein is SEQ ID NO:9, SEQ ID NO:23, SEQ ID NO:32, SEQ ID NO:47, SEQ ID NO:58, SEQ ID NO:90, SEQ ID NO:93, SEQ ID NO:102, SEQ ID NO:118, SEQ ID NO:131, SEQ ID NO:136, SEQ ID NO:139, SEQ ID NO:141, SEQ ID NO:143, SEQ ID NO:149, SEQ ID NO:152, SEQ ID NO:160, SEQ ID NO:164, SEQ ID NO:183 or SEQ ID NO:185.

In another aspect, a method of determining an expression level of an LPA-associated disease marker RNA set forth in Table 3 or Table 4 in a subject that has or is at risk for developing an LPA-associated disease is provided. The method includes obtaining a biological sample from the subject and determining an expression level of an LPA-associated disease marker RNA set forth in Table 3 or Table 4 in the biological sample.

In another aspect, a method of determining an expression level of an LPA-associated disease marker RNA of SEQ ID NO:203-499 in a subject that has or is at risk for developing an LPA-associated disease is provided. The method includes (i) obtaining a biological sample from the subject; and (ii) determining an expression level of an LPA-associated disease marker RNA of SEQ ID NO:203-499 in the biological sample.

For the methods of determining an expression level of an LPA-associated diseases marker RNA, the determining may include (a) contacting the LPA-associated disease marker RNA with a marker RNA binding agent in the biological sample, thereby forming a disease marker RNA-binding agent complex; and (b) detecting the disease marker RNA-binding agent complex. In embodiments, the marker RNA binding agent includes a detectable moiety. In embodiments, the marker RNA binding agent includes a capturing moiety. In embodiments, the capturing moiety is a cleavable capturing moiety. In embodiments, the detecting includes contacting the disease marker RNA-binding agent complex with a capturing agent, thereby forming a captured disease marker RNA-binding agent complex. In embodiments, the detecting includes (1) contacting the captured disease marker RNA-binding agent complex with a tagging moiety; thereby forming a tagged disease marker RNA-binding agent complex; and (2) separating the tagged disease marker RNA-binding agent complex from the biological sample. In embodiments, the detecting further includes after the separating of step (2) separating the capturing binding agent from the tagged disease marker RNA-binding agent complex, thereby forming a cleaved disease marker RNA-binding agent complex. In embodiments, the detecting includes (3) separating the marker RNA binding agent from the cleaved disease marker RNA-binding agent complex; thereby forming a released marker RNA binding agent; and (4) determining an amount of the released marker RNA binding agent.

In embodiments, the method includes selecting a subject that has or is at risk for developing an LPA-associated disease. In embodiments, the biological sample is a blood-derived biological sample, a urine-derived biological sample or a saliva-derived biological sample of the subject. In embodiments, the blood-derived biological sample is whole blood, serum or plasma.

In embodiments, the method includes administering to the subject an effective amount of a modulator of the LPA-associated disease marker RNA set forth in Table 3 or Table 4. In embodiments, the method includes administering to the subject an effective amount of a modulator of the LPA-associated disease marker RNA of SEQ ID NO:203-499. In embodiments, the modulator is an antagonist. In embodiments, the antagonist is a peptide, small molecule, nucleic acid, antibody or aptamer. In embodiments, the expression level of the LPA-associated disease marker RNA set forth in Table 3 or Table 4 is elevated relative to a standard control. In embodiments, the expression level of the LPA-associated disease marker RNA of SEQ ID NO:203-499 is elevated relative to a standard control. In embodiments, the modulator is an agonist. In embodiments, the agonist is a peptide, small molecule, nucleic acid, antibody or aptamer. In embodiments, the expression level of the LPA-associated disease marker RNA set forth in Table 3 or Table 4 is decreased relative to a standard control. In embodiments, the expression level of the LPA-associated disease marker RNA of SEQ ID NO:203-499 is decreased relative to a standard control. In embodiments, the method includes administering to the subject an effective amount of a further therapeutic agent.

In embodiments, the LPA-associated disease marker RNA is SEQ ID NO:209, SEQ ID NO:214, SEQ ID NO:216, SEQ ID NO:217, SEQ ID NO:218, SEQ ID NO:219, SEQ ID NO:222, SEQ ID NO:236, SEQ ID NO:237, SEQ ID NO:242, SEQ ID NO:243, SEQ ID NO:267, SEQ ID NO:269, SEQ ID NO:270, SEQ ID NO:271, SEQ ID NO:303, SEQ ID NO:309, SEQ ID NO:353, SEQ ID NO:364, SEQ ID NO:378, SEQ ID NO:396, SEQ ID NO:417, SEQ ID NO:432 or SEQ ID NO:448.

In another aspect, a method of determining whether a subject has or is at risk of developing an LPA-associated disease is provided. The method includes (i) detecting an expression level of one or more LPA-associated disease marker proteins set forth in Table 1 or Table 2 in a subject. (ii) It is determined whether the expression level is increased or decreased relative to a standard control, wherein an elevated expression level of an LPA-associated disease marker protein set forth in Table 1 or Table 2 or a decreased expression level of an LPA-associated disease marker protein set forth in Table 1 or Table 2 relative to the standard control indicates that the subject has or is at risk of developing an LPA-associated disease. Based at least in part on the expression level in step (ii), it is determined whether the subject has or is at risk for developing an LPA-associated disease.

In another aspect, a method of determining whether a subject has or is at risk of developing an LPA-associated disease is provided. The method includes (i) detecting an expression level of one or more LPA-associated disease marker proteins of SEQ ID NO:1-202 in a subject. (ii) It is determined whether the expression level is increased or decreased relative to a standard control, wherein an elevated expression level of an LPA-associated disease marker protein of SEQ ID NO:1-202 or a decreased expression level of an LPA-associated disease marker protein of SEQ ID NO:1-202 relative to the standard control indicates that the subject has or is at risk of developing an LPA-associated disease. And (iii) based at least in part on the expression level in step (ii), determining whether the subject has or is at risk for developing an LPA-associated disease.

In embodiments, the method includes selecting a subject that has or is at risk for developing an LPA-associated disease. In embodiments, the expression level of one or more LPA-associated disease marker proteins set forth in Table 1 or Table 2 is detected from a biological sample of the subject. In embodiments, the expression level of one or more LPA-associated disease marker proteins of SEQ ID NO:1-202 is detected from a biological sample of the subject. In embodiments, the biological sample is a blood-derived biological sample, a urine-derived biological sample or a saliva-derived biological sample of the subject. In embodiments, the blood-derived biological sample is whole blood, serum or plasma. In embodiments, the method includes administering to the subject an effective amount of a modulator of the LPA-associated disease marker protein set forth in Table 1 or Table 2. In embodiments, the method includes administering to the subject an effective amount of a modulator of the LPA-associated disease marker protein of SEQ ID NO:1-202. In embodiments, the modulator is an antagonist. In embodiments, the antagonist is a peptide, small molecule, nucleic acid, antibody or aptamer. In embodiments, the modulator is an agonist. In embodiments, the agonist is a peptide, small molecule, nucleic acid, antibody or aptamer. In embodiments, the method includes administering to the subject an effective amount of a further therapeutic agent.

In another aspect, a method of determining whether a subject has or is at risk of developing an LPA-associated disease is provided. The method includes (i) detecting an expression level of one or more LPA-associated disease marker RNAs set forth in Table 3 or Table 4 in a subject. (ii) It is determined whether the expression level is increased or decreased relative to a standard control, wherein an elevated expression level of an LPA-associated disease marker RNA set forth in Table 3 or Table 4 or a decreased expression level of an LPA-associated disease marker RNA set forth in Table 3 or Table 4 relative to the standard control indicates that the subject has or is at risk of developing an LPA-associated disease. (iii) Based at least in part on the expression level in step (ii), it is determined whether the subject has or is at risk for developing an LPA-associated disease.

In another aspect, a method of determining whether a subject has or is at risk of developing an LPA-associated disease is provided. The method includes (i) detecting an expression level of one or more LPA-associated disease marker RNAs of SEQ ID NO:203-499 in a subject. (ii) It is determined whether the expression level is increased or decreased relative to a standard control, wherein an elevated expression level of an LPA-associated disease marker RNA of SEQ ID NO:203-499 or a decreased expression level of an LPA-associated disease marker RNA of SEQ ID NO:203-499 relative to the standard control indicates that the subject has or is at risk of developing an LPA-associated disease. And (iii) based at least in part on the expression level in step (ii), determining whether the subject has or is at risk for developing an LPA-associated disease.

In embodiments, the method includes selecting a subject that has or is at risk for developing an LPA-associated disease. In embodiments, the expression level of one or more LPA-associated disease marker RNAs set forth in Table 3 or Table 4 is detected from a biological sample of the subject. In embodiments, the expression level of one or more LPA-associated disease marker RNAs of SEQ ID NO:203-499 is detected from a biological sample of the subject. In embodiments, the biological sample is a blood-derived biological sample, a urine-derived biological sample or a saliva-derived biological sample of the subject. In embodiments, the blood-derived biological sample is whole blood, serum or plasma. In embodiments, the method includes administering to the subject an effective amount of a modulator of the LPA-associated disease marker RNA set forth in Table 3 or Table 4. In embodiments, the method includes administering to the subject an effective amount of a modulator of the LPA-associated disease marker RNA of SEQ ID NO:203-499. In embodiments, the modulator is an antagonist. In embodiments, the antagonist is a peptide, small molecule, nucleic acid, antibody or aptamer. In embodiments, the modulator is an agonist. In embodiments, the agonist is a peptide, small molecule, nucleic acid, antibody or aptamer. In embodiments, the method includes administering to the subject an effective amount of a further therapeutic agent.

In another aspect, a method of determining an LPA-associated disease activity in a patient is provided. The method includes (i) determining a first expression level of a protein set forth in Table 1 or Table 2 in the patient at a first time point. (ii) A second expression level of a protein set forth in Table 1 or Table 2 in the patient is determined at a second time point. (iii) The second expression level of a protein set forth in Table 1 or Table 2 is compared to the first expression level of a protein set forth in Table 1 or Table 2, thereby determining the LPA-associated disease activity in the patient.

In another aspect, a method of determining an LPA-associated disease activity in a patient is provided. The method includes (i) determining a first expression level of a protein of SEQ ID NO:1-202 in the patient at a first time point. (ii) A second expression level of a protein of SEQ ID NO:1-202 in the patient is determined at a second time point. (iii) The second expression level of a protein of SEQ ID NO:1-202 is compared to the first expression level of a protein of SEQ ID NO:1-202, thereby determining the LPA-associated disease activity in the patient.

In another aspect, a method of determining an LPA-associated disease activity in a patient is provided. The method includes (i) determining a first expression level of an RNA set forth in Table 3 or Table 4 in the patient at a first time point. (ii) A second expression level of an RNA set forth in Table 3 or Table 4 is determined in the patient at a second time point. (iii) The second expression level of an RNA set forth in Table 3 or Table 4 is compared to the first expression level of an RNA set forth in Table 3 or Table 4, thereby determining the LPA-associated disease activity in the patient.

In another aspect, a method of determining an LPA-associated disease activity in a patient is provided. The method includes (i) determining a first expression level of an RNA of SEQ ID NO:203-499 in the patient at a first time point. (ii) A second expression level of an RNA of SEQ ID NO:203-499 is determined in the patient at a second time point. (iii) The second expression level of an RNA of SEQ ID NO:203-499 is compared to the first expression level of an RNA of SEQ ID NO:203-499, thereby determining the LPA-associated disease activity in the patient.

In another aspect, a method of determining whether an LPA-associated disease patient is at risk for progression of the LPA-associated disease is provided. The method includes (i) detecting an expression level of one or more LPA-associated disease marker proteins set forth in Table 1 or Table 2 in an LPA-associated disease patient. (ii) It is determined whether the expression level is modulated relative to a standard control, wherein a modulated expression level of an LPA-associated disease marker protein set forth in Table 1 or Table 2 relative to the standard control indicates that the LPA-associated disease patient is at risk for progression of the LPA-associated disease. (iii) Based at least in part on the expression level in step (ii), it is determined whether the LPA-associated disease patient is at risk for progression of the LPA-associated disease.

In another aspect, a method of determining whether an LPA-associated disease patient is at risk for progression of the LPA-associated disease is provided. The method includes (i) detecting an expression level of one or more LPA-associated disease marker proteins set forth of SEQ ID NO:1-202 in an LPA-associated disease patient. (ii) It is determined whether the expression level is modulated relative to a standard control, wherein a modulated expression level of an LPA-associated disease marker protein of SEQ ID NO:1-202 relative to the standard control indicates that the LPA-associated disease patient is at risk for progression of the LPA-associated disease; and (iii) based at least in part on the expression level in step (ii), determining whether the LPA-associated disease patient is at risk for progression of the LPA-associated disease.

In another aspect, a method of determining whether an LPA-associated disease patient is at risk for progression of the LPA-associated disease is provided. The method includes (i) detecting an expression level of one or more LPA-associated disease marker RNAs set forth in Table 3 or Table 4 in an LPA-associated disease patient. (ii) It is determined whether the expression level is modulated relative to a standard control, wherein a modulated expression level of an LPA-associated disease marker RNA set forth in Table 3 or Table 4 relative to the standard control indicates that the LPA-associated disease patient is at risk for progression of the LPA-associated disease. (iii) Based at least in part on the expression level in step (ii), it is determined whether the LPA-associated disease patient is at risk for progression of the LPA-associated disease.

In another aspect, a method of determining whether an LPA-associated disease patient is at risk for progression of the LPA-associated disease is provided. The method includes (i) detecting an expression level of one or more LPA-associated disease marker RNAs of SEQ ID NO:203-499 in an LPA-associated disease patient. (ii) It is determined whether said expression level is modulated relative to a standard control, wherein a modulated expression level of an LPA-associated disease marker RNA of SEQ ID NO:203-499 relative to the standard control indicates that the LPA-associated disease patient is at risk for progression of said LPA-associated disease; and (iii) based at least in part on the expression level in step (ii), determining whether the LPA-associated disease patient is at risk for progression of the LPA-associated disease.

In another aspect, a method of determining whether an LPA-associated disease patient is at risk for progression of the LPA-associated disease is provided. The method includes (i) determining a first expression level of a protein set forth in Table 1 or Table 2 in the patient at a first time point. (ii) A second expression level of a protein set forth in Table 1 or Table 2 in the patient is determined at a second time point. (iii) The second expression level of a protein set forth in Table 1 or Table 2 is compared to the first expression level of a protein set forth in Table 1 or Table 2, wherein when the second expression level of a protein set forth in Table 1 or Table 2 is different from the first level of a protein set forth in Table 1 or Table 2, the patient is at risk for progression of the LPA-associated disease.

In another aspect, a method of determining whether an LPA-associated disease patient is at risk for progression of the LPA-associated disease is provided. The method includes (i) determining a first expression level of a protein of SEQ ID NO:1-202 in the patient at a first time point. (ii) A second expression level of a protein of SEQ ID NO:1-202 in the patient is determined at a second time point. (iii) The second expression level of a protein of SEQ ID NO:1-202 is compared to the first expression level of a protein of SEQ ID NO:1-202, wherein when the second expression level of a protein of SEQ ID NO:1-202 is different from the first level of a protein of SEQ ID NO:1-202, the patient is at risk for progression of the LPA-associated disease. T

In another aspect, a method of determining whether an LPA-associated disease patient is at risk for progression of the LPA-associated disease is provided. The method includes (i) determining a first expression level of an RNA set forth in Table 3 or Table 4 in the patient at a first time point. (ii) A second expression level of an RNA set forth in Table 3 or Table 4 in the patient is determined at a second time point. (iii) The second expression level of an RNA set forth in Table 3 or Table 4 is compared to the first expression level of an RNA set forth in Table 3 or Table 4, wherein when the second expression level of an RNA set forth in Table 3 or Table 4 is different from the first level of an RNA set forth in Table 3 or Table 4, the patient is at risk for progression of the LPA-associated disease.

In another aspect, a method of determining whether an LPA-associated disease patient is at risk for progression of the LPA-associated disease is provided. The method includes (i) determining a first expression level of an RNA of SEQ ID NO:203-499 in the patient at a first time point. (ii) A second expression level of an RNA of SEQ ID NO:203-499 in the patient is determined at a second time point. (iii) The second expression level of an RNA of SEQ ID NO:203-499 is compared to the first expression level of an RNA of SEQ ID NO:203-499, wherein when the second expression level of an RNA of SEQ ID NO:203-499 is different from the first level of an RNA of SEQ ID NO:203-499, the patient is at risk for progression of the LPA-associated disease.

The patient at risk for progression of the LPA-associated disease may have received treatment for the LPA-associated disease prior to the determining in step (i). Where the patient at risk for progression of the LPA-associated disease has received treatment for the LPA-associated disease prior to the determining in step (i) the treatment may be altered after the determining in step (i) and before the determining in step (ii). Alternatively, the patient at risk for progression of the LPA-associated disease may not have received treatment for the LPA-associated disease prior to the determining in step (i). Where the patient at risk for progression of the LPA-associated disease has not received treatment for the LPA-associated disease prior to the determining in step (i) the patient may receive treatment after the determining in step (i). Thus, in some embodiments, the method includes administering an LPA-associated disease treatment after the determining in step (i). In some embodiments, the method further includes determining a rate of progression of the LPA-associated disease in the patient based on the comparing.

In embodiments, the determining the first expression level of a protein of SEQ ID NO:1-202 or an RNA of SEQ ID NO:203-499 and the second expression level of a protein of SEQ ID NO:1-202 or an RNA of SEQ ID NO:203-499 includes normalizing the first expression level of a protein of SEQ ID NO:1-202 or an RNA of SEQ ID NO:203-499 and the second expression level of a protein of SEQ ID NO:1-202 or an RNA of SEQ ID NO:203-499 to a protein or RNA expressed from a standard gene in the patient. In some further embodiments, the standard gene is a so-called housekeeping gene, as is commonly known in the art, such as GAPDH or beta-actin. In embodiments, the standard gene is non-differentially expressed. Where the standard gene is non-differentially expressed, the expression level of the standard gene remains unchanged over the time course of the disease. In some embodiment, the first expression level is detected from a first biological sample of the subject and the second expression level is detected from a second biological sample of the subject. In some embodiment, the first biological sample is a first bodily fluid sample and the second biological sample is a second bodily fluid sample.

### III. Methods of Treatment

In another aspect, a method of treating an LPA-associated disease in a subject in need thereof is provided. The method includes administering to the subject an effective amount of an modulator of an LPA-associated disease marker protein set forth in Table 1 or Table 2, thereby treating an LPA-associated disease in the subject.

In another aspect, a method of treating an LPA-associated disease in a subject in need thereof is provided. The method includes administering to the subject an effective amount of an modulator of an LPA-associated disease marker protein of SEQ ID NO:1-202, thereby treating an LPA-associated disease in the subject.

In another aspect, a method of treating an LPA-associated disease in a subject in need thereof is provided. The method includes administering to the subject an effective amount of an modulator of an LPA-associated disease marker RNA set forth in Table 3 or Table 4, thereby treating an LPA-associated disease in the subject.

In another aspect, a method of treating an LPA-associated disease in a subject in need thereof is provided. The method includes administering to the subject an effective amount of an modulator of an LPA-associated disease marker RNA of SEQ ID NO:203-499, thereby treating an LPA-associated disease in the subject.

In another aspect, a method of treating an LPA-associated disease in a subject in need thereof is provided. The method includes (i) determining whether a subject expresses an elevated level or a decreased level of an LPA-associated disease marker protein set forth in Table 1 or Table 2 relative to a standard control. (ii) When an elevated expression level or a decreased expression level of the LPA-associated disease marker protein set forth in Table 1 or Table 2 is found relative to the standard control, an LPA-associated disease treatment, an antagonist or an agonist of an LPA-associated disease marker protein set forth in Table 1 or Table 2 is administered to the subject, thereby treating the subject.

In another aspect, a method of treating an LPA-associated disease in a subject in need thereof is provided. The method includes (i) determining whether a subject expresses an elevated level or a decreased level of an LPA-associated disease marker protein of SEQ ID NO:1-202 relative to a standard control. And (ii) when an elevated expression level or a decreased expression level of the LPA-associated disease marker protein of SEQ ID NO:1-202 is found relative to the standard control, administering to the subject an LPA-associated disease treatment, an antagonist or an agonist of an LPA-associated disease marker protein of SEQ ID NO:1-202, thereby treating the subject.

In some embodiments, the determining whether the expression level is modulated relative to a standard control includes determining whether the expression level is elevated or suppressed relative to other LPA-associated disease patients. Therefore a standard control as referred to herein may include or may be an average value gathered from a population of LPA-associated disease patients. In other embodiments, a standard control is an average value gathered from a population of normal patients.

In another aspect, a method of treating an LPA-associated disease in a subject in need thereof is provided. The method includes (i) determining whether a subject expresses an elevated level or a decreased level of an LPA-associated disease marker RNA set forth in Table 3 or Table 4 relative to a standard control. (ii) When an elevated expression level or a decreased expression level of the LPA-associated disease marker RNA set forth in Table 3 or Table 4 is found relative to the standard control, an LPA-associated disease treatment, an antagonist or an agonist of an LPA-associated disease marker RNA set forth in Table 3 or Table 4 is administered to the subject, thereby treating the subject.

In another aspect, a method of treating an LPA-associated disease in a subject in need thereof is provided. The method includes (i) determining whether a subject expresses an elevated level or a decreased level of an LPA-associated disease marker RNA of SEQ ID NO:203-499 relative to a standard control. And (ii) when an elevated expression level or a decreased expression level of the LPA-associated disease marker RNA of SEQ ID NO:203-499 is found relative to the standard control, administering to the subject an LPA-associated disease treatment, an antagonist or an agonist of an LPA-associated disease marker RNA of SEQ ID NO:203-499, thereby treating the subject.

### IV. Kits and Compositions

The invention provides kits for detection of LPA-associated disease marker proteins or LPA-associated disease marker RNAs or fragments thereof in a subject. The kit can be for personal use or provided to medical professionals. The kit can be a kit for diagnosing or prognosing and LPA-associated disease, or for monitoring the progression of disease or the efficacy of treatment.

In one aspect, a kit is provided. The kit includes (a) a marker protein binding agent (e.g., an aptamer, optionally labeled) capable of binding to a substance within a biological sample (e.g., whole blood, serum or plasma) from a human subject having or at risk of developing an LPA-associated disease; wherein the substance is an LPA-associated disease marker protein of SEQ ID NO:1-202 or fragment thereof. And (b) a detecting reagent or a detecting apparatus capable of indicating binding of the marker protein binding agent to the substance.

In another aspect, a kit is provided. The kit includes (a) a marker RNA binding agent capable of binding to a substance within a biological sample from a human subject having or at risk of developing an LPA-associated disease; wherein the substance is an LPA-associated disease marker RNA of SEQ ID NO:203-499 or fragment thereof. And (b) a detecting reagent or a detecting apparatus capable of indicating binding of the marker RNA binding agent to the substance.

The kits provided herein can further include assay containers (tubes), buffers, or enzymes necessary for carrying out the detection assay. In embodiments, the kit further includes a sample collection device for collecting a sample from a subject. In embodiments, the human subject has an LPA-associated disease.

In embodiments, the kit includes components to examine more than one LPA-associated disease marker protein or RNA or fragment thereof. For example, the kit can include more than one marker protein binding agent or marker RNA binding agent capable of binding to one or more LPA-associated disease marker proteins or fragments thereof of SEQ ID NO:1-202 or one or more LPA-associated disease marker RNAs or fragments thereof of SEQ ID NO:203-499. In embodiments, the kit includes a plurality of marker protein binding agents or a plurality of marker RNA binding agents. Where the kit includes a plurality of marker protein binding agents or marker RNA binding agents a plurality of LPA-associated disease marker proteins or RNAs or fragments thereof (e.g., of SEQ ID NO:1-202 or SEQ ID NO:203-499) are detected.

The kit will generally include at least one vial, test tube, flask, bottle, syringe or other container means, into which the testing agent, can be suitably reacted or aliquoted. Kits can also include components for comparing results such as a suitable control sample, for example a positive and/or negative control. The kit can also include a collection device for collecting and/or holding the sample from the subject. The collection device can include a sterile swab or needle (for collecting blood), and/or a sterile tube (e.g., for holding the swab or a bodily fluid sample).

In another aspect, a complex in vitro is provided. The complex includes a marker protein binding agent bound to a LPA-associated disease marker protein of SEQ ID NO:1-202 or fragment thereof, wherein the LPA-associated disease marker protein is extracted from a human subject having or at risk of developing an LPA-associated disease.

In another aspect, a complex in vitro is provided. The complex includes a marker RNA binding agent bound to a LPA-associated disease marker RNA of SEQ ID NO:203-499 or fragment thereof, wherein the LPA-associated disease marker RNA is extracted from a human subject having or at risk of developing an LPA-associated disease.

### V. Methods of Detection

The methods provided herein include the step of determining (detecting) an expression level of an LPA-associated disease marker protein or fragment thereof or an LPA-associated disease marker RNA or fragment thereof. Methods for detecting and identifying proteins or RNAs and their interactions with other proteins or nucleic acid molecules involve conventional molecular biology, microbiology, and recombinant DNA techniques within the skill of the art. Such techniques are explained fully in the literature (see, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

“Determining an expression level of a protein”, “determining a level of expression of a protein”, “determining an expression level of an RNA” or “determining a level of expression of an RNA” as provided herein includes methods and technologies well known in the art. For example, capture arrays for expression profiling may be used to determine an expression level of a protein or an RNA. Capture arrays employ high affinity capture reagents, such as conventional antibodies, single domains, engineered scaffolds, peptides, nucleic acid aptamers or complementary nucleic acids (e.g., RNA or DNA), to bind and detect specific target ligands in high throughput manner. Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; Biolnvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) are optionally useful in arrays.

The term scaffold refers to ligand-binding domains of proteins, which are engineered into multiple variants capable of binding diverse target molecules with antibody-like properties of specificity and affinity. The variants are produced in a genetic library format and selected against individual targets by phage, bacterial or ribosome display. Such ligand-binding scaffolds or frameworks include Affibodies based on S. aureus protein A (Affibody, Bromma, Sweden), Trinectins based on fibronectins (Phylos, Lexington, Mass.) and Anticalins based on the lipocalin structure (Pieris Proteolab, Freising-Weihenstephan, Germany). These are used on capture arrays in a similar fashion to antibodies and have advantages of robustness and ease of production.

Nonprotein capture molecules, notably the nucleic acid aptamers which bind protein ligands with high specificity and affinity, are also used in arrays (SomaLogic, Boulder, Colo.). Aptamers are selected from libraries of oligonucleotides by the Selex™ procedure (SomaLogic, Boulder, Colo.) and their interaction with protein is enhanced by covalent attachment, through incorporation of brominated deoxyuridine and UV-activated crosslinking (photoaptamers). Photocrosslinking to ligand reduces the cross reactivity of aptamers due to the specific steric requirements. Aptamers have the advantages of ease of production by automated oligonucleotide synthesis and the stability and robustness of DNA; on photoaptamer arrays, universal fluorescent protein stains are used to detect binding. Other nonprotein capture molecules include complementary nucleic acid molecules (e.g., RNA, DNA) capable of hybridizing to the marker RNA. In embodiments, the complementary nucleic acid molecules are attached to a detectable moiety.

Protein analytes binding to antibody arrays are detected directly or indirectly, for example, via a secondary antibody. Direct labeling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately. Serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through molecular imprinting technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which is useful diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumor extracts, and SELDI-TOF mass spectrometry is used to detection the retained proteins.

Large-scale functional chips have been constructed by immobilizing large numbers of purified proteins and are used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g., via a His tag and immobilized. Cell free protein transcription/translation is a viable alternative for synthesis of proteins which do not express well in bacterial or other in vivo systems.

In embodiments, determining (detecting) an expression level of an LPA-associated disease marker protein or fragment thereof as provided herein includes contacting an LPA-associated disease marker protein with a marker protein binding agent. A “marker protein binding agent” as provided herein refers to a substance capable of binding an LPA-associated disease marker protein. The marker protein binding agent may be a nucleic acid or a protein. In embodiments, the marker protein binding agent is an aptamer. In embodiments, the marker protein binding agent is a peptide. In embodiments, the marker protein binding agent is a small molecule. In embodiments, the marker protein binding agent is an antibody. In embodiments, the LPA-associated disease marker protein or fragment thereof is contacted with a marker protein binding agent in a biological sample (e.g., whole blood, serum or plasma). In embodiments, the marker protein binding agent includes a detectable moiety. In embodiments, the detectable moiety is a fluorescent moiety. In embodiments, the marker protein binding agent includes a capturing moiety. A “capturing moiety” refers to a protein or nucleic acid, which is covalently, through a linker or a chemical bond, or noncovalently attached to the marker protein binding agent and is capable of interacting with a capturing agent. An example of a capturing moiety useful for the methods provided herein is biotin. In embodiments, the capturing moiety is biotin. In embodiments, the capturing moiety is a cleavable capturing moiety. In embodiments, the capturing moiety is photocleavable biotin.

In embodiments, determining (detecting) an expression level of an LPA-associated disease marker RNA or fragment thereof as provided herein includes contacting LPA-associated disease marker RNA with a marker RNA binding agent. A “marker RNA binding agent” as provided herein refers to a substance capable of binding an LPA-associated disease marker RNA. The marker RNA binding agent may be a nucleic acid or a protein. In embodiments, the marker RNA binding agent is an aptamer. In embodiments, the marker RNA binding agent is a peptide. In embodiments, the marker RNA binding agent is a small molecule. In embodiments, the marker RNA binding agent is an antibody. In embodiments, the LPA-associated disease marker RNA or fragment thereof is contacted with a marker RNA binding agent in a biological sample (e.g., whole blood, serum or plasma). In embodiments, the marker RNA binding agent includes a detectable moiety. In embodiments, the detectable moiety is a fluorescent moiety. In embodiments, the marker RNA binding agent includes a capturing moiety. A “capturing moiety” refers to a protein or nucleic acid, which is covalently, through a linker or a chemical bond, or noncovalently attached to the marker RNA binding agent and is capable of interacting with a capturing agent. An example of a capturing moiety useful for the methods provided herein is biotin. In embodiments, the capturing moiety is biotin. In embodiments, the capturing moiety is a cleavable capturing moiety. In embodiments, the capturing moiety is photocleavable biotin.

A “capturing agent” as provided herein refers to an agent capable of binding a capturing moiety. The interaction between the capturing moiety and the capturing agent may be a high affinity interaction, wherein the capturing moiety and the capturing agent bind to each other (e.g., biotin, streptavidin). An example of a capturing agent useful for the methods provided herein are streptavidin coated beads. In embodiments, the capturing agent is a streptavidin coated bead. Without limitation any suitable affinity binding pairs known in the art may be used as capturing moiety and capturing agent in the methods provided herein. For example, the capturing moiety may be an antibody and the capturing agent may be an antigen-coated bead. In embodiments, the capturing moiety is biotin and the capturing agent is a streptavidin coated bead.

The marker protein binding agent may bind non-covalently to the LPA-associated disease marker protein through ionic, van der Waals, electrostatic or hydrogen bonds. Upon binding of the marker protein binding agent to the LPA-associated disease marker protein a disease marker-protein binding agent complex is formed. The methods provided herein including embodiments thereof include detecting the disease marker-protein binding agent complex, thereby determining the expression level of a LPA-associated disease marker protein or fragment thereof in a biological sample. Thus, in embodiments, the determining includes (a) contacting an LPA-associated disease marker protein with a marker protein binding agent in the biological sample, thereby forming a disease marker protein-binding agent complex; and (b) detecting the disease marker protein-binding agent complex. The disease marker protein-binding agent complex may be separated from the sample and unbound components contained therein by contacting the disease marker protein-binding agent complex with a capturing agent as described above (e.g., streptavidin-coated beads). Thus, in embodiments, the detecting includes contacting the disease marker protein-binding agent complex with a capturing agent, thereby forming a captured disease marker protein-binding agent complex. The captured disease marker protein-binding agent complex may be washed to remove any unbound components.

The marker RNA binding agent may bind non-covalently to the LPA-associated disease marker RNA through ionic, van der Waals, electrostatic or hydrogen bonds. Upon binding of the marker RNA binding agent to the LPA-associated disease marker RNA a disease marker-RNA binding agent complex is formed. The methods provided herein including embodiments thereof include detecting the disease marker-RNA binding agent complex, thereby determining the expression level of an LPA-associated disease marker RNA or fragment thereof in a biological sample. Thus, in embodiments, the determining includes (a) contacting an LPA-associated disease marker RNA with a marker RNA binding agent in the biological sample, thereby forming a disease marker RNA-binding agent complex; and (b) detecting the disease marker RNA-binding agent complex. The disease marker RNA-binding agent complex may be separated from the sample and unbound components contained therein by contacting the disease marker RNA-binding agent complex with a capturing agent as described above (e.g., streptavidin-coated beads). Thus, in embodiments, the detecting includes contacting the disease marker RNA-binding agent complex with a capturing agent, thereby forming a captured disease marker RNA-binding agent complex. The captured disease marker RNA-binding agent complex may be washed to remove any unbound components.

For the methods provided herein, the LPA-associated disease marker protein or fragment thereof or an LPA-associated disease marker RNA may be contacted with a tagging moiety. A “tagging moiety” as provided herein is a composition capable of non-covalently binding to the LPA-associated disease marker protein or RNA or fragment thereof. In embodiments, the tagging moiety is biotin. Upon binding to the LPA-associated disease marker protein or RNA or fragment thereof, the tagging moiety may bind through high affinity interaction with a tagging agent (e.g., streptavidin). In embodiments, the LPA-associated disease marker protein or RNA or fragment thereof is contacted with a tagging moiety after the formation of a captured disease marker protein-binding agent complex. In embodiments, the LPA-associated disease marker protein or fragment thereof is contacted with a tagging moiety before the formation of a captured disease marker protein-binding agent complex. In embodiments, the LPA-associated disease marker protein or fragment thereof is contacted with a tagging moiety at the same time as the formation of a captured disease marker protein-binding agent complex. In embodiments, the detecting further includes (1) contacting the captured disease marker protein-binding agent complex with a tagging moiety; thereby forming a tagged disease marker protein-binding agent complex; and (2) separating the tagged disease marker protein-binding agent complex from the biological sample.

Once the tagged disease marker protein-binding agent complex is separated from the biological sample, the interaction between capturing moiety (e.g., photocleavable biotin) and capturing agent (e.g., streptavidin-coated beads) is reversed (e.g. through cleavage of the photocleavable biotin) and a cleaved disease marker protein-binding agent complex is formed. The cleaved disease marker protein-binding agent complex includes a LPA-associated disease marker protein or fragment thereof bound to a marker protein binding agent and a tagging moiety. Thus, in embodiments, the detecting further includes after the separating of step (2) separating the capturing binding agent from the tagged disease marker protein-binding agent complex, thereby forming a cleaved disease marker protein-binding agent complex.

The cleaved disease marker protein-binding agent complex may be contacted with a tagging agent (e.g., streptavidin-coated beads) and the tagging moiety (e.g., biotin) bound to the LPA-associated disease marker protein or fragment thereof may form a high affinity interaction with the tagging agent. The cleaved disease marker protein-binding agent complex may be captured by a tagging agent (e.g., streptavidin-coated beads) and the marker protein binding agent may be subsequently separated, (e.g., eluted by affinity chromatography) from the cleaved disease marker protein-binding agent complex. Once the marker protein binding agent (e.g., aptamer) is released (separated) from the cleaved disease marker protein-binding agent complex it may be quantified using standard techniques know in the art to quantify labeled nucleic acids molecules (e.g., hybridization to a custom DNA microarray). Thus, in embodiments, the detecting further includes (3) separating the marker protein binding agent from the cleaved disease marker protein-binding agent complex; thereby forming a released marker protein binding agent; and (4) determining an amount of released marker protein binding agent.

### VI. Marker Proteins and RNAs

The LPA-associated disease marker proteins and LPA-associated disease marker RNAs as provided herein (e.g., set forth in Table 1, 2, 3, 4 or 5) are applicable to all methods, kits and compositions described herein. For the methods, kits and compositions described herein the expression level of one or more LPA-associated disease marker proteins or LPA-associated disease marker RNAs may be determined (detected). In embodiments, the expression level of at least one LPA-associated disease marker protein or LPA-associated disease marker RNA is determined (detected). In embodiments, the expression level of a plurality (e.g., 2, 3, 4, 5, 6, 7, 8, 9, 10, 11, 12, 13, 14, 15, 16, 17, 18, 19, or 20) of LPA-associated disease marker proteins or LPA-associated disease marker RNAs is determined (detected). Where the expression level of an LPA-associated disease marker protein or an LPA-associated disease marker RNA is determined (detected), the expression level of a combination of any one of the LPA-associated disease marker proteins or LPA-associated disease marker RNAs provided herein is determined (detected).

For the methods, kits and compositions described herein the expression level of one or more LPA-associated disease marker proteins and one or more LPA-associated disease marker RNAs may be determined (detected). In embodiments, the expression level of at least one LPA-associated disease marker protein and at least one LPA-associated disease marker RNA is determined (detected). In embodiments, the expression level of a plurality (e.g., 2, 3, 4, 5, 6, 7, 8, 9, 10, 11, 12, 13, 14, 15, 16, 17, 18, 19, or 20) of LPA-associated disease marker proteins and a plurality (e.g., 2, 3, 4, 5, 6, 7, 8, 9, 10, 11, 12, 13, 14, 15, 16, 17, 18, 19, or 20) of LPA-associated disease marker RNAs is determined (detected). For example, the expression level of a combination of LPA-associated disease marker proteins set forth in Table 1 or 2 or of SEQ ID NO:1-202 may be determined (detected) in combination with one or more LPA-associated disease marker RNAs set forth in Table 3 or 4 or of SEQ ID NO:203-499.

For example, the expression level of a combination of LPA-associated disease marker proteins set forth in Table 1 or 2 or of SEQ ID NO:1-202 may be determined (detected) in the methods, kits or compositions provided herein. In embodiments, the LPA-associated disease marker protein is CXCL3/CXCL2, PAI-1, ANGPT1, P41, GREM1, NID2, NET4, NXPH1, THBS1, TIMP-3, BASI, CAPG, CTAP-III, DDR2, IGFBP-2, MMP-1, MMP-9, NAP-2, CCLS or SERPINE2. In embodiments, the LPA-associated disease marker protein is CXCL3/CXCL2. In embodiments, the LPA-associated disease marker protein is PAI-1. In embodiments, the LPA-associated disease marker protein is ANGPT1. In embodiments, the LPA-associated disease marker protein is P41. In embodiments, the LPA-associated disease marker protein is GREM1. In embodiments, the LPA-associated disease marker protein is NID2. In embodiments, the LPA-associated disease marker protein is NET4. In embodiments, NXPH1. In embodiments, the LPA-associated disease marker protein is THBS1. In embodiments, the LPA-associated disease marker protein is TIMP-3. In embodiments, the LPA-associated disease marker protein is BASI. In embodiments, the LPA-associated disease marker protein is CAPG. In embodiments, the LPA-associated disease marker protein is CTAP-III. In embodiments, the LPA-associated disease marker protein is DDR2. In embodiments, the LPA-associated disease marker protein is IGFBP-2. In embodiments, the LPA-associated disease marker protein is MMP-1. In embodiments, the LPA-associated disease marker protein is MMP-9. In embodiments, the LPA-associated disease marker protein is NAP-2. In embodiments, the LPA-associated disease marker protein is CCL5. In embodiments, the LPA-associated disease marker protein is SERPINE2.

In embodiments, the expression level of CXCL3/CXCL2 and one or more LPA-associated disease marker proteins as provided herein (e.g., set forth in Table 1 or 2 or of SEQ ID NO:1-202) is determined (detected). In embodiments, the expression level of PAI-1 and one or more LPA-associated disease marker proteins as provided herein (e.g., set forth in Table 1 or 2 or of SEQ ID NO:1-202) is determined (detected). In embodiments, the expression level of ANGPT1 and one or more LPA-associated disease marker proteins as provided herein (e.g., set forth in Table 1 or 2 or of SEQ ID NO:1-202) is determined (detected). In embodiments, the expression level of P41 and one or more LPA-associated disease marker proteins as provided herein (e.g., set forth in Table 1 or 2 or of SEQ ID NO:1-202) is determined (detected). In embodiments, the expression level of GREM1 and one or more LPA-associated disease marker proteins as provided herein (e.g., set forth in Table 1 or 2 or of SEQ ID NO:1-202) is determined (detected). In embodiments, the expression level of NID2 and one or more LPA-associated disease marker proteins as provided herein (e.g., set forth in Table 1 or 2 or of SEQ ID NO:1-202) is determined (detected). In embodiments, the expression level of NET4 and one or more LPA-associated disease marker proteins as provided herein (e.g., set forth in Table 1 or 2 or of SEQ ID NO:1-202) is determined (detected). In embodiments, the expression level of NXPH1 and one or more LPA-associated disease marker proteins as provided herein (e.g., set forth in Table 1 or 2 or of SEQ ID NO:1-202) is determined (detected).

In embodiments, the expression level of THBS1 and one or more LPA-associated disease marker proteins as provided herein (e.g., set forth in Table 1 or 2 or of SEQ ID NO:1-202) is determined (detected). In embodiments, the expression level of TIMP-3 and one or more LPA-associated disease marker proteins as provided herein (e.g., set forth in Table 1 or 2 or of SEQ ID NO:1-202) is determined (detected). In embodiments, the expression level of BASI and one or more LPA-associated disease marker proteins as provided herein (e.g., set forth in Table 1 or 2 or of SEQ ID NO:1-202) is determined (detected). In embodiments, the expression level of CAPG and one or more LPA-associated disease marker proteins as provided herein (e.g., set forth in Table 1 or 2 or of SEQ ID NO:1-202) is determined (detected). In embodiments, the expression level of CTAP-III and one or more LPA-associated disease marker proteins as provided herein (e.g., set forth in Table 1 or 2 or of SEQ ID NO:1-202) is determined (detected). In embodiments, the expression level of DDR2 and one or more LPA-associated disease marker proteins as provided herein (e.g., set forth in Table 1 or 2 or of SEQ ID NO:1-202) is determined (detected).

In embodiments, the expression level of IGFBP-2 and one or more LPA-associated disease marker proteins as provided herein (e.g., set forth in Table 1 or 2 or of SEQ ID NO:1-202) is determined (detected). In embodiments, the expression level of MMP-1 and one or more LPA-associated disease marker proteins as provided herein (e.g., set forth in Table 1 or 2 or of SEQ ID NO:1-202) is determined (detected). In embodiments, the expression level of MMP-9 and one or more LPA-associated disease marker proteins as provided herein (e.g., set forth in Table 1 or 2 or of SEQ ID NO:1-202) is determined (detected). In embodiments, the expression level of NAP-2 and one or more LPA-associated disease marker proteins as provided herein (e.g., set forth in Table 1 or 2 or of SEQ ID NO:1-202) is determined (detected). In embodiments, the expression level of CCL5 and one or more LPA-associated disease marker proteins as provided herein (e.g., set forth in Table 1 or 2 or of SEQ ID NO:1-202) is determined (detected). In embodiments, the expression level of SERPINE2 and one or more LPA-associated disease marker proteins as provided herein (e.g., set forth in Table 1 or 2 or of SEQ ID NO:1-202) is determined (detected).

For example, the expression level of a combination of LPA-associated disease marker RNAs set forth in Table 3 or 4 or of SEQ ID NO:203-499 may be determined (detected) in the methods, kits or compositions provided herein. In embodiments, the LPA-associated disease marker RNA is CYR61, KRTAP1-5, DKK1, MCP-1/CCL-2, HBEGF, CTGF, KPRP, IL6TNFAIP3, KRTAP4-12, BIRC3, MIR218-1, VGLL3, IER3, GPR37, DUSP1, EFNB2, HAS2, PDCD1LG2, SERPINB2, UGCG, CHIC2, PTX3, CNST or TXNIP. In embodiments, the LPA-associated disease marker RNA is CYR61. In embodiments, the LPA-associated disease marker RNA is KRTAP1-5. In embodiments, the LPA-associated disease marker RNA is DKK1. In embodiments, the LPA-associated disease marker RNA is MCP-1/CCL-2. In embodiments, the LPA-associated disease marker RNA is HBEGF. In embodiments, the LPA-associated disease marker RNA is CTGF. In embodiments, the LPA-associated disease marker RNA is KPRP. In embodiments, the LPA-associated disease marker RNA is IL6TNFAIP3. In embodiments, the LPA-associated disease marker RNA is KRTAP4-12. In embodiments, the LPA-associated disease marker RNA is BIRC3. In embodiments, the LPA-associated disease marker RNA is MIR218-1.

In embodiments, the LPA-associated disease marker RNA is VGLL3. In embodiments, the LPA-associated disease marker RNA is IER3. In embodiments, the LPA-associated disease marker RNA is GPR37. In embodiments, the LPA-associated disease marker RNA is DUSP1. In embodiments, the LPA-associated disease marker RNA is EFNB2. In embodiments, the LPA-associated disease marker RNA is HAS2. In embodiments, the LPA-associated disease marker RNA is PDCD1LG2. In embodiments, the LPA-associated disease marker RNA is SERPINB2. In embodiments, the LPA-associated disease marker RNA is UGCG. In embodiments, the LPA-associated disease marker RNA is CHIC2. In embodiments, the LPA-associated disease marker RNA is PTX3. In embodiments, the LPA-associated disease marker RNA is CNST. In embodiments, the LPA-associated disease marker RNA is TXNIP.

In embodiments, the expression level of CYR61 and one or more LPA-associated disease marker RNAs as provided herein (e.g., set forth in Table 3 or 4 or of SEQ ID NO:203-499) is determined (detected). In embodiments, the expression level of KRTAP1-5 and one or more LPA-associated disease marker RNAs as provided herein (e.g., set forth in Table 3 or 4 or of SEQ ID NO:203-499) is determined (detected). In embodiments, the expression level of DKK1 and one or more LPA-associated disease marker RNAs as provided herein (e.g., set forth in Table 3 or 4 or of SEQ ID NO:203-499) is determined (detected). In embodiments, the expression level of MCP-1/CCL-2 and one or more LPA-associated disease marker RNAs as provided herein (e.g., set forth in Table 3 or 4 or of SEQ ID NO:203-499) is determined (detected).

In embodiments, the expression level of HBEGF and one or more LPA-associated disease marker RNAs as provided herein (e.g., set forth in Table 3 or 4 or of SEQ ID NO:203-499) is determined (detected). In embodiments, the expression level of CTGF and one or more LPA-associated disease marker RNAs as provided herein (e.g., set forth in Table 3 or 4 or of SEQ ID NO:203-499) is determined (detected). In embodiments, the expression level of KPRP and one or more LPA-associated disease marker RNAs as provided herein (e.g., set forth in Table 3 or 4 or of SEQ ID NO:203-499) is determined (detected). In embodiments, the expression level of IL6TNFAIP3 and one or more LPA-associated disease marker RNAs as provided herein (e.g., set forth in Table 3 or 4 or of SEQ ID NO:203-499) is determined (detected). In embodiments, the expression level of KRTAP4-12 and one or more LPA-associated disease marker RNAs as provided herein (e.g., set forth in Table 3 or 4 or of SEQ ID NO:203-499) is determined (detected). In embodiments, the expression level of BIRC3 and one or more LPA-associated disease marker RNAs as provided herein (e.g., set forth in Table 3 or 4 or of SEQ ID NO:203-499) is determined (detected). In embodiments, the expression level of MIR218-1 and one or more LPA-associated disease marker RNAs as provided herein (e.g., set forth in Table 3 or 4 or of SEQ ID NO:203-499) is determined (detected).

In embodiments, the expression level of VGLL3 and one or more LPA-associated disease marker RNAs as provided herein (e.g., set forth in Table 3 or 4 or of SEQ ID NO:203-499) is determined (detected). In embodiments, the expression level of IER3 and one or more LPA-associated disease marker RNAs as provided herein (e.g., set forth in Table 3 or 4 or of SEQ ID NO:203-499) is determined (detected). In embodiments, the expression level of GPR37 and one or more LPA-associated disease marker RNAs as provided herein (e.g., set forth in Table 3 or 4 or of SEQ ID NO:203-499) is determined (detected). In embodiments, the expression level of DUSP1 and one or more LPA-associated disease marker RNAs as provided herein (e.g., set forth in Table 3 or 4 or of SEQ ID NO:203-499) is determined (detected). In embodiments, the expression level of EFNB2 and one or more LPA-associated disease marker RNAs as provided herein (e.g., set forth in Table 3 or 4 or of SEQ ID NO:203-499) is determined (detected). In embodiments, the expression level of HAS2 and one or more LPA-associated disease marker RNAs as provided herein (e.g., set forth in Table 3 or 4 or of SEQ ID NO:203-499) is determined (detected). In embodiments, the expression level of PDCD1LG2 and one or more LPA-associated disease marker RNAs as provided herein (e.g., set forth in Table 3 or 4 or of SEQ ID NO:203-499) is determined (detected). In embodiments, the expression level of SERPINB2 and one or more LPA-associated disease marker RNAs as provided herein (e.g., set forth in Table 3 or 4 or of SEQ ID NO:203-499) is determined (detected).

In embodiments, the expression level of UGCG and one or more LPA-associated disease marker RNAs as provided herein (e.g., set forth in Table 3 or 4 or of SEQ ID NO:203-499) is determined (detected). In embodiments, the expression level of CHIC2 and one or more LPA-associated disease marker RNAs as provided herein (e.g., set forth in Table 3 or 4 or of SEQ ID NO:203-499) is determined (detected). In embodiments, the expression level of PTX3 and one or more LPA-associated disease marker RNAs as provided herein (e.g., set forth in Table 3 or 4 or of SEQ ID NO:203-499) is determined (detected). In embodiments, the expression level of CNST F and one or more LPA-associated disease marker RNAs as provided herein (e.g., set forth in Table 3 or 4 or of SEQ ID NO:203-499) is determined (detected). In embodiments, the expression level of TXNIP and one or more LPA-associated disease marker RNAs as provided herein (e.g., set forth in Table 3 or 4 or of SEQ ID NO:203-499) is determined (detected).

### VII. Specific Embodiments

LPA Receptor Expression in Cells

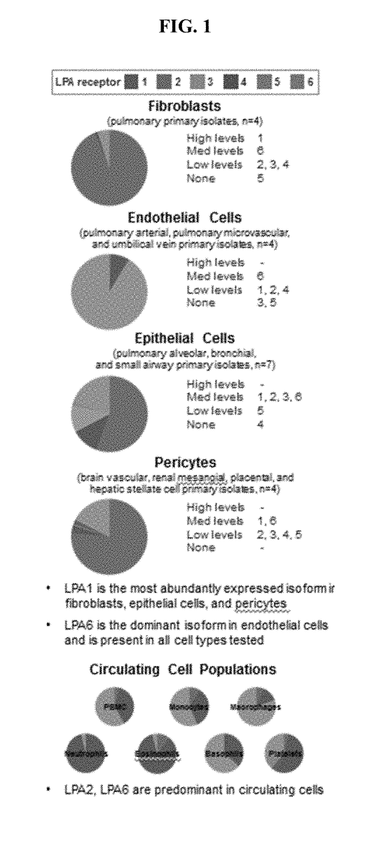

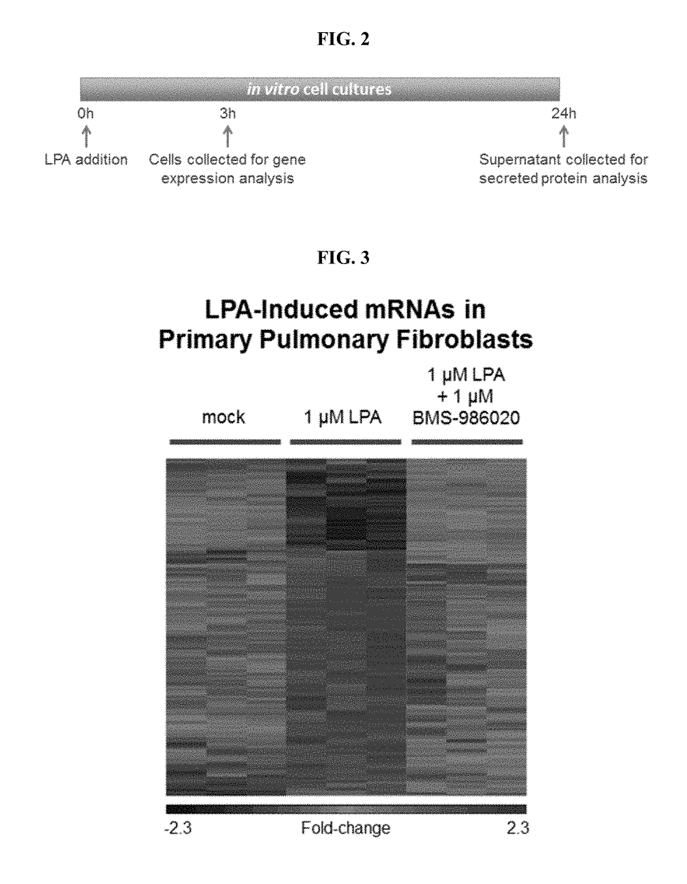

Expression of LPA receptors 1-6 was assayed by qRT-PCR in primary human fibroblasts, endothelial cells, epithelial cells, and pericytes. LPA1 is the most abundantly expressed isoform in fibroblasts, epithelial cells, and pericytes. LPA6 is the dominant isoform in endothelial cells and is present in all cell types tested and LPA2, LPA6 are predominant in circulating cells (FIG. 1).

Experimental Design for Gene Expression and Secreted Protein Analysis

Changes in gene expression after 3 hours of LPA induction were profiled on Affymetrix Human Gene ST 2.0 microarrays; differentially expressed genes identified by ANOVA with FDR <0.05 and fold-change >1.2. Process networks associated with differentially expressed genes were defined using MetaCore. Changes in secreted protein expression after 24 hours of LPA induction were profiled on an aptamer-based proteomic platform (1129 proteins); differentially expressed proteins identified by t-test.

Gene Expression after LPA Induction

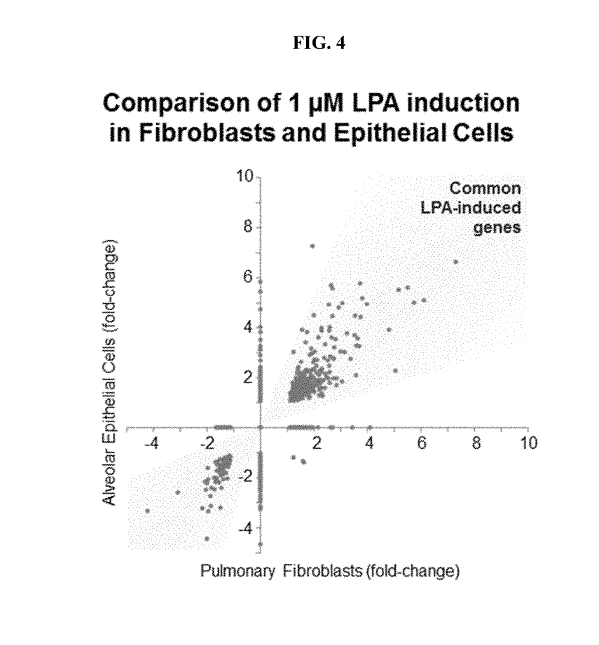

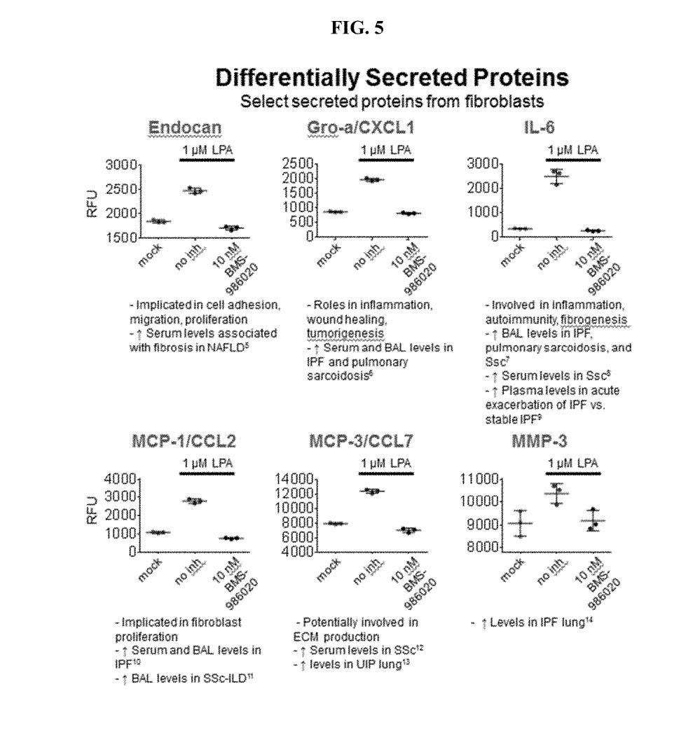

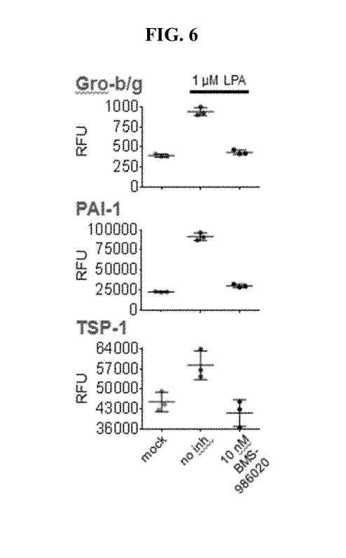

FIG. 4 shows a comparison of 1 μM LPA induction in fibroblasts and epithelial cells. LPA induced significant changes in gene expression in fibroblasts and epithelial cells as shown in Tables 3 and 4. Changes were broadly inhibited by addition of a selective LPA1 receptor antagonist. LPA-induced both common and cell-specific gene expression changes in fibroblasts and epithelial cells as shown in Tables 3 and 4. LPA-induced pathways include without limitation, cytoskeletal remodeling and immune response in fibroblasts and epithelial cells, cell adhesion in fibroblasts, and epithelial-mesenchymal transition in epithelial cells. LPA induced changes in secreted protein expression in fibroblasts and epithelial cells are shown in Tables 1, 2, and 5. Changes could be inhibited by an LPA1 receptor antagonist. Several LPA-induced proteins were observed as differentially expressed in plasma of IPF patients compared to controls (Table 5).

LPA1 is significantly expressed in cells relevant to fibrosis, including fibroblasts, epithelial cells, and pericytes. LPA6 is predominant in endothelial cells, but is present in all cell types tested. Unbiased characterization of gene and secreted protein expression identified common, as well as cell-specific, effects. LPA-induced genes and proteins were associated with pathways implicated in fibrosis. Several have also been independently associated with IPF. Treatment with a selective LPA1 receptor antagonist inhibited LPA-dependent changes.

Treatment of Cells with LPA, DNA Isolation and mRNA Expression Analysis of Marker RNAs Shown in Table 3 and 4:

Total RNA was isolated from normal human lung fibroblasts, human bronchial epithelial cells, and human pulmonary alveolar epithelial cells treated for 4 hours with LPA and/or LPAR1 inhibitor. Each treatment condition was assayed in triplicate. Samples were profiled on Affymetrix Human Gene ST Arrays 1.0 and/or 2.0 after preparation using the Ambion WT Expression Kit according to manufacturers' instructions.

mRNA Expression Data Analysis of Marker RNAs Shown in Table 3 and 4

Data normalization and all subsequent analyses were performed in Partek Genomics Suite (Partek, St. Louis, Mo.). Data were normalized using the Robust Multi-array Average (RMA) algorithm. Differentially expressed genes were identified by ANOVA with correction for multiple comparisons with p-value with FDR<0.05. A fold-change cut-off was not strictly applied, although the majority of the differentially expressed genes identified have fold-changes greater than 1.2. Genes were included if they were statistically significant for (1) LPA-induction and (2) response to treatment with an LPAR1 inhibitor.

Protein Analysis

Treatment of cells with LPA, isolation supernatant, analysis of secreted protein expression using soma panel was performed as follows: Supernatant was collected from in vitro cultures of normal human lung fibroblasts, human bronchial epithelial cells, and human pulmonary alveolar epithelial cells treated for 24 hours with LPA and/or LPAR1 inhibitor. Each treatment condition was assayed in triplicate. 1129 proteins were profiled using a broad aptamer-based proteomic platform (SomaLogic, Colo., US). Briefly, the technology uses DNA aptamers containing chemically modified nucleotides as highly specific protein binding reagents in a multiplexed assay that transforms the quantity of each targeted protein into a corresponding quantify of the aptamer. Protein quantities are then detected on a microarray platform and recorded as Relative Fluorescence Units (RFU).

In Tables 1-5 The differentially expressed secreted proteins set forth in Tables 1, 2, and 5 and the differentially expressed mRNAs set forth in Table 3 and 4 were identified by visual inspection of individual dot-plots.

Cell Culture Conditions

For the gene and protein expression analysis primary fibroblasts (NHLF product #CC-2512 from Lonza group LTD) and primary epithelial cells (NHBE Product #CC-2540 from Lonza; HPAEpiC Product #3200 from Sciencell (USA, California)) were cultured as recommended by the manufacturer, except they were cultured on collagen coated plates. As part of the induction protocol cells were starved overnight in low (0.2 or 0.1% serum) or no serum (0%), prior to stimulation with LPA.

### VIII. References

1. Shea B S and Tager A M. Pro Am Thorac Soc 2012. 9(3):102-110.
2. Swaney J S et al. BJP 2010. 160:1699-1713.
3. Montesi S B et al. BMC Pulm Med 2014. 14:5.
4. Tokumura A et al. Int J Med Sci 2009. 6(4):168-176.
5. Elsheikh E et al. Hepatology 2013. 58(S1):92A-207A.
6. Antoniou K M et al. Chest 2006. 130(4):982-988.
7. Takizawa H et al. Clin Exp Immunol 1997. 107(1):175-181.
8. Needleman B W et al. Arthr Rheumat 1992. 35:67-72.
9. Collard H R et al. Am J Physiol Lung Cell Mol Physiol 2010. 299(1):L3-7.
10. Suga M et al. Eur Resp J 1999. 14(2):376-382.
11. Caporali R et al. Sarcoidosis Vasc Diffuse Lung Dis 2004. 21(2):111-118.
12. Yanaba K et al. Ann Rheum Dis 2006. 65(1):124-126.
13. Choi E S et al. Am J Respir Crit Care Med 2004. 170(5):508-515.
14. Yamashita C M et al. Am J Pathol 2011. 179(4):1733-45.

### IX. Tables

TABLE 1
Secreted Proteins

| SEQ | | | | | | effect of |
|---|---|---|---|---|---|---|
| ID | | | | | effect | LPAR1 |
| NO: | Target | UniProt | EntrezGeneID | EntrezGeneSymbol | of LPA | inhibitor |
| 1 | 41 | P11171 | 2035 | EPB41 | up | down |
| 2 | Activin A | P08476 | 3624 | INHBA | up | down |
| 3 | ADAM 9 | Q13443 | 8754 | ADAM9 | up | down |
| 4 | ADAM12 | O43184 | 8038 | ADAM12 | | down |
| 5 | Aflatoxin B1 | O43488 | 8574 | AKR7A2 | | up |
| | aldehyde | | | | | |
| | reductase | | | | | |
| 6 | ALCAM | Q13740 | 214 | ALCAM | up | down |
| 7 | Aminoacylase-1 | Q03154 | 95 | ACY1 | up | up |
| 8 | Angiogenin | P03950 | 283 | ANG | up | down |
| 9 | Angiopoietin-1 | Q15389 | 284 | ANGPT1 | up | down |
| 10 | Angiopoietin-2 | O15123 | 285 | ANGPT2 | down | up |
| 11 | Angiostatin | P00747 | 5340 | PLG | down | |
| 12 | ANGL4 | Q9BY76 | 51129 | ANGPTL4 | up | |
| 13 | annexin I | P04083 | 301 | ANXA1 | up | |
| 14 | Apo D | P05090 | 347 | APOD | | up |
| 15 | Apo E2 | P02649 | 348 | APOE | down | |
| 16 | Apo E3 | P02649 | 348 | APOE | down | |
| 17 | AREG | P15514 | 374 | AREG | up | |
| 18 | ART | O00253 | 181 | AGRP | | up |
| 19 | ARTS1 | Q9NZ08 | 51752 | ERAP1 | up | down |
| 20 | ASM3A | Q92484 | 10924 | SMPDL3A | up | |
| 21 | ATS1 | Q9UHI8 | 9510 | ADAMTS1 | down | up |
| 22 | B7-H1 | Q9NZQ7 | 29126 | CD274 | up | down |
| 23 | BASI | P35613 | 682 | BSG | up | |
| 24 | BFL1 | Q16548 | 597 | BCL2A1 | up | down |
| 25 | BGH3 | Q15582 | 7045 | TGFBI | up | down |
| 26 | BGN | P21810 | 633 | BGN | up | down |
| 27 | Bone | P07585 | 1634 | DCN | up | down |
| | proteoglycan II | | | | | |
| 28 | BSSP4 | Q9GZN4 | 64063 | PRSS22 | up | down |
| 29 | C1r | P00736 | 715 | C1R | | up |
| 30 | Cadherin-2 | P19022 | 1000 | CDH2 | | up |
| 31 | calgranulin B | P06702 | 6280 | S100A9 | | up |
| 32 | CAPG | P40121 | 822 | CAPG | up | up |
| 33 | Carbonic | P23280 | 765 | CA6 | | up |
| | anhydrase 6 | | | | | |
| 34 | Caspase-3 | P42574 | 836 | CASP3 | up | up |
| 35 | Cathepsin A | P10619 | 5476 | CTSA | up | down |
| 36 | Cathepsin H | P09668 | 1512 | CTSH | up | down |
| 37 | Cathepsin S | P25774 | 1520 | CTSS | up | down |
| 38 | Cathepsin V | O60911 | 1515 | CTSL2 | up | down |
| 39 | CATZ | Q9UBR2 | 1522 | CTSZ | up | down |
| 40 | CK2-A1:B | P68400 | 1457 | CSNK2A1 | up | |
| | | P67870 | 1460 | CSNK2B | | |
| 41 | Coactosin-like | Q14019 | 23406 | COTL1 | | down |
| | protein | | | | | |
| 42 | Coagulation | P00742 | 2159 | F10 | down | up |
| | Factor Xa | | | | | |
| 43 | Cofilin-1 | P23528 | 1072 | CFL1 | up | |
| 44 | COMMD7 | Q86VX2 | 149951 | COMMD7 | up | |
| 45 | CRDL1 | Q9BU40 | 91851 | CHRDL1 | up | down |
| 46 | CRP | P02741 | 1401 | CRP | | down |
| 47 | CTAP-III | P02775 | 5473 | PPBP | up | down |
| 48 | CXCL16 soluble | Q9H2A7 | 58191 | CXCL16 | up | |
| 49 | Cyclophilin A | P62937 | 5478 | PPIA | up | up |
| 50 | Cystatin M | Q15828 | 1474 | CST6 | up | down |
| 51 | DAF | P08174 | 1604 | CD55 | up | down |
| 52 | DBNL | Q9UJU6 | 28988 | DBNL | up | down |
| 53 | DcR3 | O95407 | 8771 | TNFRSF6B | up | down |
| 54 | DC-SIGN | Q9NNX6 | 30835 | CD209 | | down |
| 55 | DERM | Q07507 | 1805 | DPT | down | up |
| 56 | Desmoglein-1 | Q02413 | 1828 | DSG1 | | up |
| 57 | discoidin domain | Q08345 | 780 | DDR1 | up | down |
| | receptor 1 | | | | | |
| 58 | Discoidin domain | Q16832 | 4921 | DDR2 | up | down |
| | receptor 2 | | | | | |
| 59 | DKK1 | O94907 | 22943 | DKK1 | up | down |
| 60 | DKK3 | Q9UBP4 | 27122 | DKK3 | up | down |
| 61 | Dkk-4 | Q9UBT3 | 27121 | DKK4 | up | down |
| 62 | DLRB1 | Q9NP97 | 83658 | DYNLRB1 | | up |
| 63 | DPP2 | Q9UHL4 | 29952 | DPP7 | up | down |
| 64 | eIF-5A-1 | P63241 | 1984 | EIF5A | down | up |
| 65 | Elafin | P19957 | 5266 | PI3 | up | |
| 66 | EMR2 | Q9UHX3 | 30817 | EMR2 | up | down |
| 67 | ENA-78 | P42830 | 6374 | CXCL5 | up | down |
| 68 | Endocan | Q9NQ30 | 11082 | ESM1 | up | down |
| 69 | Endothelin- | P42892 | 1889 | ECE1 | up | down |
| | converting | | | | | |
| | enzyme 1 | | | | | |
| 70 | Eotaxin | P51671 | 6356 | CCL11 | up | down |
| 71 | EPHA3 | P29320 | 2042 | EPHA3 | up | down |
| 72 | EphB4 | P54760 | 2050 | EPHB4 | up | |
| 73 | Ephrin-A5 | P52803 | 1946 | EFNA5 | up | down |
| 74 | Ephrin-B3 | Q15768 | 1949 | EFNB3 | up | down |
| 75 | EPI | O14944 | 2069 | EREG | up | down |
| 76 | Epithelial cell | P29317 | 1969 | EPHA2 | up | down |
| | kinase | | | | | |
| 77 | FCAR | P24071 | 2204 | FCAR | down | |
| 78 | FGF-18 | O76093 | 8817 | FGF18 | down | up |
| 79 | FGF-20 | Q9NP95 | 26281 | FGF20 | up | down |
| 80 | FGF7 | P21781 | 2252 | FGF7 | up | down |
| 81 | Flt-3 | P36888 | 2322 | FLT3 | up | up |
| 82 | FST | P19883 | 10468 | FST | up | down |
| 83 | FSTL3 | O95633 | 10272 | FSTL3 | up | down |
| 84 | Galectin-3 | P17931 | 3958 | LGALS3 | up | up |
| 85 | GAPDH liver | P04406 | 2597 | GAPDH | up | up |
| 86 | GCP-2 | P80162 | 6372 | CXCL6 | up | down |
| 87 | G-CSF | P09919 | 1440 | CSF3 | up | down |
| 88 | GDF-9 | O60383 | 2661 | GDF9 | down | up |
| 89 | gp130 soluble | P40189 | 3572 | IL6ST | up | down |
| 90 | GREM1 | O60565 | 26585 | GREM1 | up | down |
| 91 | GRN | P28799 | 2896 | GRN | up | down |
| 92 | Gro-a | P09341 | 2919 | CXCL1 | up | down |
| 93 | Gro-b/g | P19876 | None | CXCL3 CXCL2 | up | down |
| | | P19875 | | | | |
| 94 | GV | P39877 | 5322 | PLA2G5 | up | down |
| 95 | HAI-1 | O43278 | 6692 | SPINT1 | | up |
| 96 | HGF | P14210 | 3082 | HGF | up | down |
| 97 | Histone H2A.z | P0C0S5 | 3015 | H2AFZ | | down |
| 98 | hnRNP A/B | Q99729 | 3182 | HNRNPAB | | up |
| 99 | HNRPQ | O60506 | 10492 | SYNCRIP | | up |
| 100 | ICOS | Q9Y6W8 | 29851 | ICOS | | down |
| 101 | IDE | P14735 | 3416 | IDE | up | |
| 102 | IGFBP-2 | P18065 | 3485 | IGFBP2 | down | up |
| 103 | IGFBP-3 | P17936 | 3486 | IGFBP3 | up | down |
| 104 | IGFBP-7 | Q16270 | 3490 | IGFBP7 | up | down |
| 105 | IGF-I sR | P08069 | 3480 | IGF1R | up | down |
| 106 | IGF-II receptor | P11717 | 3482 | IGF2R | up | down |
| 107 | IL-1 R4 | Q01638 | 9173 | IL1RL1 | up | down |
| 108 | IL-11 | P20809 | 3589 | IL11 | up | down |
| 109 | IL-12 RB2 | Q99665 | 3595 | IL12RB2 | up | down |
| 110 | IL-16 | Q14005 | 3603 | IL16 | | up |
| 111 | IL-17D | Q8TAD2 | 53342 | IL17D | up | down |
| 112 | IL-6 | P05231 | 3569 | IL6 | up | down |
| 113 | IL-6 sRa | P08887 | 3570 | IL6R | up | down |
| 114 | IL-8 | P10145 | 3576 | IL8 | up | down |
| 115 | IP-10 | P02778 | 3627 | CXCL10 | up | down |
| 116 | JAG1 | P78504 | 182 | JAG1 | up | down |
| 117 | JAG2 | Q9Y219 | 3714 | JAG2 | up | down |
| 118 | KPCI | P41743 | 5584 | PRKCI | up | |
| 119 | Layilin | Q6UX15 | 143903 | LAYN | up | down |
| 120 | LG3BP | Q08380 | 3959 | LGALS3BP | up | down |
| 121 | LGMN | Q99538 | 5641 | LGMN | up | down |
| 122 | LRIG3 | Q6UXM1 | 121227 | LRIG3 | up | down |
| 123 | MATN2 | O00339 | 4147 | MATN2 | | down |
| 124 | MCP-1 | P13500 | 6347 | CCL2 | up | down |
| 125 | MCP-3 | P80098 | 6354 | CCL7 | up | down |
| 126 | Met | P08581 | 4233 | MET | up | down |
| 127 | MICA | Q29983 | 4276 | MICA | up | down |
| 128 | MICB | Q29980 | 4277 | MICB | up | down |
| 129 | Midkine | P21741 | 4192 | MDK | up | |
| 130 | MIP-3a | P78556 | 6364 | CCL20 | up | down |
| 131 | MMP-1 | P03956 | 4312 | MMP1 | up | down |
| 132 | MMP-10 | P09238 | 4319 | MMP10 | up | down |
| 133 | MMP-12 | P39900 | 4321 | MMP12 | up | down |
| 134 | MMP-13 | P45452 | 4322 | MMP13 | up | down |
| 135 | MMP-3 | P08254 | 4314 | MMP3 | up | down |
| 136 | MMP-9 | P14780 | 4318 | MMP9 | up | down |
| 137 | MRC2 | Q9UBG0 | 9902 | MRC2 | up | down |
| 138 | NAGK | Q9UJ70 | 55577 | NAGK | up | |
| 139 | NAP-2 | P02775 | 5473 | PPBP | up | down |
| 140 | NCAM-L1 | P32004 | 3897 | L1CAM | up | down |
| 141 | NET4 | Q9HB63 | 59277 | NTN4 | up | down |
| 142 | Neurotrophin-3 | P20783 | 4908 | NTF3 | up | down |
| 143 | NID2 | Q14112 | 22795 | NID2 | up | down |
| 144 | NKp46 | O76036 | 9437 | NCR1 | up | down |
| 145 | Nogo Receptor | Q9BZR6 | 65078 | RTN4R | up | down |
| 146 | NovH | P48745 | 4856 | NOV | up | down |
| 147 | NRP1 | O14786 | 8829 | NRP1 | up | down |
| 148 | NRX3B | Q9HDB5 | 9369 | NRXN3 | up | down |
| 149 | NXPH1 | P58417 | 30010 | NXPH1 | up | down |
| 150 | OMD | Q99983 | 4958 | OMD | up | down |
| 151 | ON | P09486 | 6678 | SPARC | up | down |
| 152 | PAI-1 | P05121 | 5054 | SERPINE1 | up | down |
| 153 | PARK7 | Q99497 | 11315 | PARK7 | up | down |
| 154 | PCSK7 | Q16549 | 9159 | PCSK7 | up | down |
| 155 | PDE11 | Q9HCR9 | 50940 | PDE11A | | down |
| 156 | PDGF-AA | P04085 | 5154 | PDGFA | up | down |
| 157 | PDGF-BB | P01127 | 5155 | PDGFB | up | |
| 158 | PGP9.5 | P09936 | 7345 | UCHL1 | | down |
| 159 | PHI | P06744 | 2821 | GPI | | up |
| 160 | Protease nexin I | P07093 | 5270 | SERPINE2 | up | down |
| 161 | PSD7 | P51665 | 5713 | PSMD7 | | down |
| 162 | PTHrP | P12272 | 5744 | PTHLH | up | down |
| 163 | RAC1 | P63000 | 5879 | RAC1 | up | down |
| 164 | RANTES | P13501 | 6352 | CCL5 | up | down |
| 165 | RAP | P30533 | 4043 | LRPAP1 | up | |
| 166 | RGMB | Q6NW40 | 285704 | RGMB | up | down |
| 167 | ROBO2 | Q9HCK4 | 6092 | ROBO2 | up | down |
| 168 | ROR1 | Q01973 | 4919 | ROR1 | | down |
| 169 | SARP-2 | Q8N474 | 6422 | SFRP1 | up | down |
| 170 | SCGF-alpha | Q9Y240 | 6320 | CLEC11A | up | down |
| 171 | SDF-1 | P48061 | 6387 | CXCL12 | up | down |
| 172 | Semaphorin 3A | Q14563 | 10371 | SEMA3A | up | down |
| 173 | sFRP-3 | Q92765 | 2487 | FRZB | down | up |
| 174 | SLIK5 | O94991 | 26050 | SLITRK5 | up | down |
| 175 | Sphingosine | Q9NYA1 | 8877 | SPHK1 | up | up |
| | kinase 1 | | | | | |
| 176 | Spondin-1 | Q9HCB6 | 10418 | SPON1 | down | up |
| 177 | SSRP1 | Q08945 | 6749 | SSRP1 | | up |
| 178 | ST4S6 | Q7LFX5 | 51363 | CHST15 | up | down |
| 179 | suPAR | Q03405 | 5329 | PLAUR | up | down |
| 180 | TAJ | Q9NS68 | 55504 | TNFRSF19 | up | down |
| 181 | TFPI | P10646 | 7035 | TFPI | up | down |
| 182 | TGF-b R III | Q03167 | 7049 | TGFBR3 | up | down |
| 183 | Thrombospondin- | P07996 | 7057 | THBS1 | up | down |
| | 1 | | | | | |
| 184 | TIG2 | Q99969 | 5919 | RARRES2 | up | down |
| 185 | TIMP-3 | P35625 | 7078 | TIMP3 | up | down |
| 186 | TNF sR-I | P19438 | 7132 | TNFRSF1A | up | down |
| 187 | TNF sR-II | P20333 | 7133 | TNFRSF1B | up | down |
| 188 | TNFSF15 | O95150 | 9966 | TNFSF15 | up | down |
| 189 | TrATPase | P13686 | 54 | ACP5 | down | up |
| 190 | TRY3 | P35030 | 5646 | PRSS3 | | up |
| 191 | TSG-6 | P98066 | 7130 | TNFAIP6 | up | down |
| 192 | TSLP | Q969D9 | 85480 | TSLP | up | down |
| 193 | TSP2 | P35442 | 7058 | THBS2 | up | down |
| 194 | TWEAK | O43508 | 8742 | TNFSF12 | down | up |
| 195 | TXD12 | O95881 | 51060 | TXNDC12 | up | down |
| 196 | UNC5H3 | O95185 | 8633 | UNC5C | up | down |
| 197 | uPA | P00749 | 5328 | PLAU | up | down |
| 198 | URB | Q76M96 | 151887 | CCDC80 | up | down |
| 199 | VEGF | P15692 | 7422 | VEGFA | up | down |
| 200 | VEGF121 | P15692 | 7422 | VEGFA | up | down |
| 201 | VEGF-C | P49767 | 7424 | VEGFC | up | down |
| 202 | WKFN1 | Q96D09 | 114928 | GPRASP2 | down | up |

TABLE 2
Secreted Proteins
Secreted protein overlap with plasma proteins
differentially expressed in IPF patients

| | | | IPF vs. |
|---|---|---|---|
| | | | control |
| SEQ | | | (fold- |
| ID NO: | Protein | | change) |
| 93 | CXCL3 | Gro-b/g | 1.62 |
| | CXCL2 | | |
| 152 | PAI-1 | Serpine peptidase inhibitor, clade E | 1.53 |
| 9 | ANGPT1 | Angiopoietin-1 | 1.36 |
| 118 | P41 | P41 | 1.53 |
| 90 | GREM1 | Gremlin 1 | 1.57 |
| 143 | NID2 | Nidogen 2 (osteonidogen) | 1.45 |
| 141 | NET4 | Transmembrane protein 53 | 1.28 |
| 149 | NXPH1 | Neuroexophilin 1 | 1.41 |
| 183 | THBS1 | Thrombospondin-1 | 1.47 |
| 185 | TIMP-3 | TIMP metallopeptidase inhibitor 3 | 1.43 |
| 23 | BASI | Extracellular matrix metalloproteinase | 1.76 |
| 32 | CAPG | Macrophage-capping protein | 1.90 |
| 47 | CTAP-III | Pro-platelet basic protein | 1.43 |
| 58 | DDR2 | Discoidin domain receptor tyrosine | 1.25 |
| | | kinase 2 | |
| 102 | IGFBP-2 | Insulin-like growth factor-binding | −1.27 |
| | | protein 2 | |
| 131 | MMP-1 | Matrix metallopeptidase 1 | 1.51 |
| 136 | MMP-9 | Matrix metallopeptidase 9 | 1.49 |
| 139 | NAP-2 | Napsin B aspartic peptidase | 1.53 |
| 164 | CCL5 | RANTES | 1.55 |
| 160 | SERPINE2 | protease nexin I | 1.99 |

TABLE 3
Gene expression from fibroblasts and/or epithelial cells

| | | | | effect of |
|---|---|---|---|---|
| SEQ | | | effect of | LPAR1 |
| ID NO: | Gene Symbol | RefSeq | LPA | inhibitor |
| 203 | ABL2 | NM_007314 | up | down |
| 204 | AKAP12 | NM_005100 | up | down |
| 205 | AMOTL2 | NM_016201 | up | down |
| 206 | ARRDC3 | NM_020801 | down | up |
| 207 | ARRDC4 | NM_183376 | down | up |
| 208 | BAZ1A | NM_013448 | up | down |
| 209 | BIRC3 | NM_001165 | up | down |
| 210 | C3orf52 | NM_024616 | up | down |
| 211 | C7orf49 | NR_024185 | down | up |
| 212 | CDK6 | NM_001259 | up | down |
| 213 | CDR2 | NM_001802 | up | down |
| 214 | CHIC2 | NM_012110 | up | down |
| 215 | CRIM1 | NM_016441 | up | down |
| 216 | CTGF | NM_001901 | up | down |
| 217 | CYR61 | NM_001554 | up | down |
| 218 | DKK1 | NM_012242 | up | down |
| 219 | DUSP1 | NM_004417 | up | down |
| 220 | DUSP14 | NM_007026 | up | down |
| 221 | DUSP5 | NM_004419 | up | down |
| 222 | EFNB2 | NM_004093 | up | down |
| 223 | ELL2 | NM_012081 | up | down |
| 224 | ENC1 | NM_003633 | up | down |
| 225 | EPHA2 | NM_004431 | up | down |
| 226 | ERRFI1 | NM_018948 | up | down |
| 227 | ETS1 | NM_001143820 | up | down |
| 228 | FAM86B1 | NR_003494 | down | up |
| 229 | FERMT2 | NM_006832 | up | down |
| 230 | FGF5 | NM_004464 | up | down |
| 231 | FOSB | NM_006732 | up | down |
| 232 | FOSL1 | NM_005438 | up | down |
| 233 | FST | NM_006350 | up | down |
| 234 | GADD45B | NM_015675 | up | down |
| 235 | GPCPD1 | NM_019593 | down | up |
| 236 | HAS2 | NM_005328 | up | down |
| 237 | HBEGF | NM_001945 | up | down |
| 238 | HIVEP2 | NM_006734 | up | down |
| 239 | ICAM1 | NM_000201 | up | down |
| 240 | INHBA | NM_002192 | up | down |
| 241 | JMY | NM_152405 | down | up |
| 242 | KPRP | NM_001025231 | up | down |
| 243 | KRTAP1-5 | NM_031957 | up | down |
| 244 | LIF | NM_002309 | up | down |
| 245 | LMO7 | NM_005358 | up | down |
| 246 | MAP2K3 | NM_145109 | up | down |
| 247 | MPV17L2 | NM_032683 | down | up |
| 248 | MYADM | NM_001020818 | up | down |
| 249 | NAV3 | NM_014903 | up | down |
| 250 | NFKB2 | NM_002502 | up | down |
| 251 | NFKBIZ | NM_031419 | up | down |
| 252 | PLAUR | NM_002659 | up | down |
| 253 | PRDM1 | NM_001198 | up | down |
| 254 | RAPH1 | NM_213589 | up | down |
| 255 | RASA2 | NM_006506 | up | down |
| 256 | SEMA7A | NM_003612 | up | down |
| 257 | SERPINB9 | NM_004155 | up | down |
| 258 | SERPINE1 | NM_000602 | up | down |
| 259 | SGK1 | NM_001143676 | up | down |
| 260 | SH3RF1 | NM_020870 | up | down |
| 261 | SLC38A2 | NM_018976 | up | down |
| 262 | SMURF2 | NM_022739 | up | down |
| 263 | SPOCD1 | NM_144569 | up | down |
| 264 | TAGLN3 | NM_013259 | up | down |
| 265 | THBS1 | NM_003246 | up | down |
| 266 | TMEM2 | NM_013390 | up | down |
| 267 | TNFAIP3 | NM_006290 | up | down |
| 268 | TSC22D2 | NM_014779 | up | down |
| 269 | TXNIP | NM_006472 | down | up |
| 270 | UGCG | NM_003358 | up | down |
| 271 | VGLL3 | NM_016206 | up | down |
| 272 | ACPP | NM_001099 | up | down |
| 273 | ADAMTS1 | NM_006988 | up | down |
| 274 | ADAMTS6 | NM_197941 | up | down |
| 275 | AHCTF1 | NM_015446 | up | down |
| 276 | AMACR | NM_014324 | down | up |
| 277 | ANKRD13A | NM_033121 | up | down |
| 278 | APBB3 | NM_133174 | down | up |
| 279 | ARHGAP31 | NM_020754 | up | down |
| 280 | ARHGEF9 | NM_015185 | down | up |
| 281 | ARID3B | NM_006465 | up | down |
| 282 | ARL4D | NM_001661 | down | up |
| 283 | ARL5B | NM_178815 | up | down |
| 284 | ARSJ | NM_024590 | up | down |
| 285 | ASAP1 | NM_018482 | up | down |
| 286 | ASB13 | NM_024701 | down | up |
| 287 | ATP8B1 | NM_005603 | up | down |
| 288 | ATXN7L1 | NM_020725 | down | up |
| 289 | B3GNT2 | NM_006577 | up | down |
| 290 | BCAR3 | NM_003567 | up | down |
| 291 | BCL3 | NM_005178 | up | down |
| 292 | BIRC2 | NM_001166 | up | down |
| 293 | BMP2 | NM_001200 | up | down |
| 294 | BRIX1 | NM_018321 | up | down |
| 295 | C10orf78 | NM_145247 | down | up |
| 296 | C16orf52 | NM_001164579 | up | down |
| 297 | C2CD2 | NM_015500 | up | down |
| 298 | C5orf33 | NM_001085411 | down | up |
| 299 | C6orf130 | AJ420538 | down | up |
| 300 | C6orf70 | NM_018341 | down | up |
| 301 | CALD1 | NM_033138 | up | down |
| 302 | CCDC113 | NM_014157 | down | up |
| 303 | CCL2 | NM_002982 | up | down |
| 304 | CD274 | NM_014143 | up | down |
| 305 | CDK7 | NM_001799 | up | down |
| 306 | CHST2 | NM_004267 | up | down |
| 307 | CIRBP | NM_001280 | down | up |
| 308 | CITED2 | NM_006079 | up | down |
| 309 | CNST | NM_152609 | up | down |
| 310 | COL4A5 | NM_000495 | down | up |
| 311 | CRY1 | NM_004075 | up | down |
| 312 | CSF2 | NM_000758 | up | down |
| 313 | CSGALNACT2 | NM_018590 | up | down |
| 314 | CSRP2BP | NM_020536 | down | up |
| 315 | DAB2 | NM_001343 | up | down |
| 316 | DCUN1D3 | NM_173475 | up | down |
| 317 | DDAH1 | NM_012137 | up | down |
| 318 | DDX21 | NM_004728 | up | down |
| 319 | DNAJB4 | NM_007034 | up | down |
| 320 | DOK5 | NM_018431 | up | down |
| 321 | DOT1L | NM_032482 | up | down |
| 322 | DSN1 | NM_001145316 | down | up |
| 323 | DTX4 | NM_015177 | up | down |
| 324 | DZIP1 | NM_198968 | down | up |
| 325 | EDN1 | NM_001955 | up | down |
| 326 | EHD1 | NM_006795 | up | down |
| 327 | EIF2C2 | NM_012154 | up | down |
| 328 | ELF1 | NM_172373 | up | down |
| 329 | ELK3 | NM_005230 | up | down |
| 330 | EMP1 | NM_001423 | up | down |
| 331 | ENOSF1 | NM_017512 | down | up |
| 332 | ENTPD7 | NM_020354 | up | down |
| 333 | EPM2AIP1 | NM_014805 | down | up |
| 334 | EREG | NM_001432 | up | down |
| 335 | ESM1 | NM_007036 | up | down |
| 336 | EYA1 | NM_000503 | down | up |
| 337 | FAM120C | NM_017848 | down | up |
| 338 | FAM126B | NM_173822 | up | down |
| 339 | FAM180A | NM_205855 | up | down |
| 340 | FAM46C | NM_017709 | up | down |
| 341 | FBXL4 | NM_012160 | down | up |
| 342 | FGF2 | NM_002006 | up | down |
| 343 | FGF7 | NM_002009 | up | down |
| 344 | FHL2 | NM_201555 | up | down |
| 345 | FLRT2 | NM_013231 | down | up |
| 346 | FMN1 | NM_001103184 | up | down |
| 347 | FOXN2 | NM_002158 | up | down |
| 348 | FSTL1 | NM_007085 | up | down |
| 349 | GALT | NM_000155 | down | up |
| 350 | GJC1 | NM_005497 | up | down |
| 351 | GLIPR1 | NM_006851 | up | down |
| 352 | GPR176 | NM_007223 | up | down |
| 353 | GPR37 | NM_005302 | up | down |
| 354 | GUSBP3 | NR_027386 | down | up |
| 355 | GZF1 | NM_022482 | down | up |
| 356 | H1F0 | NM_005318 | down | up |
| 357 | HDDC3 | NM_198527 | down | up |
| 358 | HIST2H2BF | NM_001024599 | down | up |
| 359 | HMGCR | NM_000859 | down | up |
| 360 | HMGCS1 | NM_001098272 | down | up |
| 361 | HMMR | NM_001142556 | down | up |
| 362 | HS6ST1 | NM_004807 | down | up |
| 363 | HSD17B7P2 | NR_003086 | down | up |
| 364 | IER3 | NM_003897 | up | down |
| 365 | IGF2BP2 | NM_006548 | up | down |
| 366 | IL6 | NM_000600 | up | down |
| 367 | IL7R | NM_002185 | up | down |
| 368 | JMJD6 | NM_001081461 | up | down |
| 369 | KCNRG | NM_173605 | down | up |
| 370 | KCTD10 | NM_031954 | up | down |
| 371 | KCTD11 | NM_001002914 | up | down |
| 372 | KCTD18 | NM_152387 | down | up |
| 373 | KIAA0586 | NM_014749 | down | up |
| 374 | KIAA0776 | NM_015323 | down | up |
| 375 | KIAA0922 | NM_001131007 | up | down |
| 376 | KIAA1377 | NM_020802 | down | up |
| 377 | KLF6 | NM_001300 | up | down |
| 378 | KRTAP4-12 | NM_031854 | up | down |
| 379 | LACTB | NM_032857 | up | down |
| 380 | LATS2 | NM_014572 | up | down |
| 381 | LIAS | NM_006859 | down | up |
| 382 | LIMD1 | NM_014240 | up | down |
| 383 | LOC100130691 | NR_026966 | down | up |
| 384 | LOC100130876 | AK130278 | up | down |
| 385 | LOC100131860 | AK097109 | down | up |
| 386 | LOC100133299 | AY358688 | up | down |
| 387 | LOC100133315 | NR_029192 | down | up |
| 388 | LOC400464 | AK127420 | down | up |
| 389 | LRCH1 | NM_001164211 | up | down |
| 390 | LRRCC1 | NM_033402 | down | up |
| 391 | MAP3K8 | NM_005204 | up | down |
| 392 | MAP4K3 | NM_003618 | up | down |
| 393 | MAST4 | NM_001164664 | up | down |
| 394 | MBLAC2 | NM_203406 | down | up |
| 395 | MBNL1 | NM_021038 | up | down |
| 396 | MIR218-1 | NR_029631 | up | down |
| 397 | MIR221 | NR_029635 | up | down |
| 398 | MRM1 | NM_024864 | down | up |
| 399 | MSH5 | NM_002441 | down | up |
| 400 | MYO1E | NM_004998 | up | down |
| 401 | NAB2 | NM_005967 | up | down |
| 402 | NAPEPLD | NM_001122838 | down | up |
| 403 | NCEH1 | NM_001146276 | up | down |
| 404 | NDEL1 | NM_001025579 | up | down |
| 405 | NEU3 | NM_006656 | down | up |
| 406 | NFAT5 | NM_138714 | up | down |
| 407 | NFKB1 | NM_003998 | up | down |
| 408 | NFKBIE | NM_004556 | up | down |
| 409 | NFX1 | NM_002504 | down | up |
| 410 | NPC1 | NM_000271 | up | down |
| 411 | NRIP1 | NM_003489 | up | down |
| 412 | NUDT12 | NM_031438 | down | up |
| 413 | NUFIP2 | NM_020772 | up | down |
| 414 | ORC5 | NM_002553 | down | up |
| 415 | PALB2 | NM_024675 | down | up |
| 416 | PATZ1 | NM_014323 | down | up |
| 417 | PDCD1LG2 | NM_025239 | up | down |
| 418 | PDP1 | NM_001161778 | up | down |
| 419 | PEA15 | NM_003768 | up | down |
| 420 | PEX1 | NM_000466 | down | up |
| 421 | PEX11B | NM_003846 | down | up |
| 422 | PIK3R3 | NM_003629 | down | up |
| 423 | PMS1 | NM_000534 | down | up |
| 424 | PPM1K | NM_152542 | down | up |
| 425 | PPP1R3B | NM_024607 | up | down |
| 426 | PPP1R3C | NM_005398 | down | up |
| 427 | PRDM6 | NM_001136239 | down | up |
| 428 | PRKCD | NM_006254 | up | down |
| 429 | PRSS22 | NM_022119 | up | down |
| 430 | PTCD2 | NM_024754 | down | up |
| 431 | PTPN12 | NM_002835 | up | down |
| 432 | PTX3 | NM_002852 | up | down |
| 433 | RAB8B | NM_016530 | up | down |
| 434 | RAI14 | NM_001145525 | up | down |
| 435 | RBMS1 | NM_016836 | up | down |
| 436 | RCOR3 | NM_001136223 | down | up |
| 437 | REV3L | NM_002912 | down | up |
| 438 | RND3 | NM_005168 | up | down |
| 439 | RNF19A | NM_183419 | up | down |
| 440 | RP2 | NM_006915 | up | down |
| 441 | RWDD2B | NM_016940 | down | up |
| 442 | SAMD4A | NM_015589 | up | down |
| 443 | SCN8A | NM_014191 | up | down |
| 444 | SDC4 | NM_002999 | up | down |
| 445 | SEC24A | NM_021982 | up | down |
| 446 | SEC24B | NM_006323 | up | down |
| 447 | SENP8 | NM_145204 | down | up |
| 448 | SERPINB2 | NM_001143818 | up | down |
| 449 | SESN1 | NM_014454 | down | up |
| 450 | SGK223 | NM_001080826 | up | down |
| 451 | SGMS2 | NM_001136258 | up | down |
| 452 | SIRT5 | NM_031244 | down | up |
| 453 | SKIL | NM_005414 | up | down |
| 454 | SLC16A14 | NM_152527 | down | up |
| 455 | SLC24A1 | NM_004727 | down | up |
| 456 | SLITRK6 | NM_032229 | down | up |
| 457 | SPOPL | NM_001001664 | up | down |
| 458 | SRBD1 | NM_018079 | down | up |
| 459 | SRF | NM_003131 | up | down |
| 460 | SRGAP1 | NM_020762 | up | down |
| 461 | ST3GAL3 | NM_174963 | down | up |
| 462 | STK17B | NM_004226 | up | down |
| 463 | STK36 | NM_015690 | down | up |
| 464 | STX11 | NM_003764 | up | down |
| 465 | STXBP5 | NM_001127715 | up | down |
| 466 | SYNE1 | NM_182961 | up | down |
| 467 | TACC2 | NM_206862 | down | up |
| 468 | TBX5 | NM_181486 | down | up |
| 469 | TK2 | NM_004614 | down | up |
| 470 | TMEM192 | NM_001100389 | down | up |
| 471 | TNFRSF12A | NM_016639 | up | down |
| 472 | TP53BP2 | NM_005426 | up | down |
| 473 | TP53INP1 | NM_033285 | down | up |
| 474 | TRIB1 | NM_025195 | up | down |
| 475 | TRIO | NM_007118 | up | down |
| 476 | TSC22D3 | NM_198057 | down | up |
| 477 | TSEN2 | NM_025265 | down | up |
| 478 | UAP1 | NM_003115 | up | down |
| 479 | USP13 | NM_003940 | down | up |
| 480 | USP40 | NM_018218 | down | up |
| 481 | USP53 | NM_019050 | up | down |
| 482 | VCL | NM_014000 | up | down |
| 483 | VEGFC | NM_005429 | up | down |
| 484 | ZBTB1 | NM_001123329 | up | down |
| 485 | ZNF143 | NM_003442 | up | down |
| 486 | ZNF18 | NM_144680 | down | up |
| 487 | ZNF25 | NM_145011 | down | up |
| 488 | ZNF331 | NM_018555 | down | up |
| 489 | ZNF362 | NM_152493 | down | up |
| 490 | ZNF43 | NM_003423 | down | up |
| 491 | ZNF469 | NM_001127464 | up | down |
| 492 | ZNF717 | NM_001128223 | down | up |
| 493 | ZNF76 | NM_003427 | down | up |
| 494 | ZNF800 | NM_176814 | up | down |
| 495 | ZNF804A | NM_194250 | up | down |
| 496 | ZNRD1-AS | AF032109 | down | up |
| 497 | ZRANB1 | NM_017580 | up | down |
| 498 | ZSWIM6 | NM_020928 | up | down |
| 499 | ZYX | NM_003461 | up | down |

TABLE 4A
Differentially expressed mRNAs (List of selected LPA-induced
genes in fibroblasts (average of 3 experiments)).

| | | | Effect of 1 μM |
|---|---|---|---|
| | | Effect of 1 | BMS-986020 |
| | | μM LPA | relative to |
| | | (fold- | induced cells |
| Gene | | change) | (fold-change) |
| CYR61 | cysteine-rich, angiogenic inducer, 61 | 6.52 | −7.53 |
| KRTAP1-5 | keratin associated protein 1-5 | 6.49 | −6.89 |
| DKK1 | dickkopf homolog 1 | 6.36 | −6.16 |
| MCP-1/ | chemokine (C-C motif) ligand 2 | 5.58 | −5.99 |
| CCL2 | | | |
| LIF | leukemia inhibitory factor | 5.15 | −5.07 |
| HBEGF | heparin-binding EGF-like growth factor | 5.11 | −5.41 |
| CTGF | connective tissue growth factor | 4.71 | −4.62 |
| KPRP | keratinocyte proline-rich protein | 4.31 | −3.81 |
| IL6 | interleukin 6 | 4.19 | −4.08 |
| TNFAIP3 | tumor necrosis factor, α-induced | 3.44 | −3.68 |
| | protein 3 | | |
| KRTAP4-12 | keratin associated protein 4-12 | 3.23 | −3.90 |
| BIRC3 | baculoviral IAP repeat-containing 3 | 3.17 | −3.33 |
| MIR218-1 | microRNA 218-1 | 3.07 | −2.82 |
| VGLL3 | vestigial like 3 | 2.96 | −2.97 |
| IER3 | immediate early response 3 | 2.94 | −3.28 |
| GPR37 | G protein-coupled receptor 37 | 2.80 | −3.13 |
| DUSP1 | dual specificity phosphatase 1 | 2.76 | −2.56 |
| EFNB2 | ephrin-B2 | 2.61 | −2.63 |
| HAS2 | hyaluronan synthase 2 | 2.61 | −2.89 |
| PDCD1LG2 | programmed cell death 1 ligand 2 | 2.52 | −2.51 |
| SERPINB2 | serpin peptidase inhibitor, clade B | 2.44 | −2.64 |
| UGCG | UDP-glucose ceramide | 2.41 | −2.52 |
| | glucosyltransferase | | |
| CHIC2 | cysteine-rich hydrophobic domain 2 | 2.36 | −2.40 |
| PTX3 | pentraxin 3 | 2.34 | −2.49 |
| PAI-1 | serpin peptidase inhibitor, clade E | 2.28 | −2.43 |
| CNST | consortin, connexin sorting protein | 2.26 | −2.32 |
| TXNIP | thioredoxin interacting protein | −3.79 | 4.41 |

TABLE 4B
Differentially expressed mRNAs as listed in Table 4A including
sequence identifiers (SEQ ID NO:) (List of selected LPA-induced
genes in fibroblasts (average of 3 experiments)).

| SEQ ID NO: | Gene Symbol |
|---|---|
| 211 | CYR61 |
| 243 | KRTAP1-5 |
| 218 | DKK1 |
| 303 | MCP-1/CCL-2 |
| 237 | HBEGF |
| 216 | CTGF |
| 242 | KPRP |
| 267 | IL6TNFAIP3 |
| 378 | KRTAP4-12 |
| 209 | BIRC3 |
| 396 | MIR218-1 |
| 271 | VGLL3 |
| 364 | IER3 |
| 353 | GPR37 |
| 219 | DUSP1 |
| 222 | EFNB2 |
| 236 | HAS2 |
| 417 | PDCD1LG2 |
| 448 | SERPINB2 |
| 270 | UGCG |
| 214 | CHIC2 |
| 432 | PTX3 |
| 309 | CNST |
| 269 | TXNIP |

TABLE 5
LPA-induced proteins independently associated with IPF.

| | | IPF vs. |
|---|---|---|
| | | control |
| | | (fold- |
| Protein | | change) |
| CXCL2/3 | Gro-b/g | 1.62 |
| ANGPT1 | Angiopoietin-1 | 1.36 |
| p41 | P41 | 1.53 |
| GREM1 | Gremlin 1 | 1.57 |
| NID2 | Nidogen 2 (osteonidogen) | 1.45 |
| NET4 | Transmembrane protein 53 | 1.28 |
| NXPH1 | Neuroexophilin 1 | 1.41 |
| PAI-1 | Serpine peptidase inhibitor, | 1.53 |
| | clade E | |
| TSP-1 | Thrombospondin-1 | 1.47 |
| TIMP-3 | TIMP metallopeptidase | 1.43 |
| | inhibitor 3 | |
| CTAP-III | Pro-platelet basic protein | 1.43 |
| DDR2 | Discoidin domain receptor | 1.25 |
| | tyrosine kinase 2 | |
| MMP-1 | Matrix metallopeptidase 1 | 1.51 |
| MMP-9 | Matrix metallopeptidase 9 | 1.49 |
| NAP-2 | Napsin B aspartic peptidase | 1.53 |

### X. Embodiments

### Embodiments I

### Embodiment 1

A method of determining an expression level of an LPA-associated disease marker protein as set forth in Table 1 or Table 2 in a subject that has or is at risk for developing an LPA-associated disease, said method comprising: (i) obtaining a biological sample from said subject; and (ii) determining an expression level of an LPA-associated disease marker protein set forth in Table 1 or Table 2 in said biological sample.

### Embodiment 2

The method of embodiment 1, wherein said determining comprises: (a) contacting a LPA-associated disease marker protein with a marker protein binding agent in said biological sample, thereby forming a disease marker protein-binding agent complex; and (b) detecting said disease marker protein-binding agent complex.

### Embodiment 3

The method of embodiment 1, further comprising selecting a subject that has or is at risk for developing an LPA-associated disease.

### Embodiment 4

The method of embodiment 1, wherein said biological sample is a blood-derived biological sample, a urine-derived biological sample or a saliva-derived biological sample of said subject.

### Embodiment 5

The method of embodiment 4, wherein said blood-derived biological sample is whole blood, serum or plasma.

### Embodiment 6

The method of embodiment 1, further comprising administering to said subject an effective amount of a modulator of said LPA-associated disease marker protein set forth in Table 1 or Table 2.

### Embodiment 7

The method of embodiment 6, wherein said modulator is an antagonist.

### Embodiment 8

The method of embodiment 7, wherein said antagonist is a peptide, small molecule, nucleic acid, antibody or aptamer.

### Embodiment 9

The method of embodiment 7, wherein said expression level of said LPA-associated disease marker protein set forth in Table 1 or Table 2 is elevated relative to a standard control.

### Embodiment 10

The method of embodiment 6, wherein said modulator is an agonist.

### Embodiment 11

The method of embodiment 10, wherein said agonist is a peptide, small molecule, nucleic acid, antibody or aptamer.

### Embodiment 12

The method of embodiment 10, wherein said expression level of said LPA-associated disease marker protein set forth in Table 1 or Table 2 is decreased relative to a standard control.

### Embodiment 13

The method of embodiment 6, further comprising administering to said subject an effective amount of a further therapeutic agent.

### Embodiment 14

A method of determining an expression level of an LPA-associated disease marker RNA set forth in Table 3 or Table 4 in a subject that has or is at risk for developing an LPA-associated disease, said method comprising: (i) obtaining a biological sample from said subject; and (ii) determining an expression level of an LPA-associated disease marker RNA set forth in Table 3 or Table 4 in said biological sample.

### Embodiment 15

The method of embodiment 14, further comprising selecting a subject that has or is at risk for developing an LPA-associated disease.

### Embodiment 16

The method of embodiment 14, wherein said biological sample is a blood-derived biological sample, a urine-derived biological sample or a saliva-derived biological sample of said subject.

### Embodiment 17

The method of embodiment 16, wherein said blood-derived biological sample is whole blood, serum or plasma.

### Embodiment 18

The method of embodiment 14, further comprising administering to said subject an effective amount of a modulator of said LPA-associated disease marker RNA set forth in Table 3 or Table 4.

### Embodiment 19

The method of embodiment 18, wherein said modulator is an antagonist.

### Embodiment 20

The method of embodiment 19, wherein said antagonist is a peptide, small molecule, nucleic acid, antibody or aptamer.

### Embodiment 21

The method of embodiment 19, wherein said expression level of said LPA-associated disease marker RNA set forth in Table 3 or Table 4 is elevated relative to a standard control.

### Embodiment 22

The method of embodiment 18, wherein said modulator is an agonist.

### Embodiment 23

The method of embodiment 22, wherein said agonist is a peptide, small molecule, nucleic acid, antibody or aptamer.

### Embodiment 24

The method of embodiment 22, wherein said expression level of said LPA-associated disease marker RNA set forth in Table 3 or Table 4 is decreased relative to a standard control.

### Embodiment 25

The method of embodiment 18, further comprising administering to said subject an effective amount of a further therapeutic agent.

### Embodiment 26

A method of determining whether a subject has or is at risk of developing an LPA-associated disease, said method comprising: (i) detecting an expression level of one or more LPA-associated disease marker proteins set forth in Table 1 or Table 2 in a subject; (ii) determining whether said expression level is increased or decreased relative to a standard control, wherein an elevated expression level of an LPA-associated disease marker protein set forth in Table 1 or Table 2 or a decreased expression level of an LPA-associated disease marker protein set forth in Table 1 or Table 2 relative to said standard control indicates that said subject has or is at risk of developing an LPA-associated disease; and (iii) based at least in part on said expression level in step (ii), determining whether said subject has or is at risk for developing an LPA-associated disease.

### Embodiment 27

The method of embodiment 26, further comprising selecting a subject that has or is at risk for developing an LPA-associated disease.

### Embodiment 28

The method of embodiment 26, wherein said expression level of one or more LPA-associated disease marker proteins set forth in Table 1 or Table 2 is detected from a biological sample of said subject.

### Embodiment 29

The method of embodiment 28, wherein said biological sample is a blood-derived biological sample, a urine-derived biological sample or a saliva-derived biological sample of said subject.

### Embodiment 30

The method of embodiment 29, wherein said blood-derived biological sample is whole blood, serum or plasma.

### Embodiment 31

The method of embodiment 26, further comprising administering to said subject an effective amount of a modulator of said LPA-associated disease marker protein set forth in Table 1 or Table 2.

### Embodiment 32

The method of embodiment 31, wherein said modulator is an antagonist.

### Embodiment 33

The method of embodiment 32, wherein said antagonist is a peptide, small molecule, nucleic acid, antibody or aptamer.

### Embodiment 34

The method of embodiment 31, wherein said modulator is an agonist.

### Embodiment 35

The method of embodiment 34, wherein said agonist is a peptide, small molecule, nucleic acid, antibody or aptamer.

### Embodiment 36

The method of embodiment 31, further comprising administering to said subject an effective amount of a further therapeutic agent.

### Embodiment 37

A method of determining whether a subject has or is at risk of developing an LPA-associated disease, said method comprising: (i) detecting an expression level of one or more LPA-associated disease marker RNAs set forth in Table 3 or Table 4 in a subject; (ii) determining whether said expression level is increased or decreased relative to a standard control, wherein an elevated expression level of an LPA-associated disease marker RNA set forth in Table 3 or Table 4 or a decreased expression level of an LPA-associated disease marker RNA set forth in Table 3 or Table 4 relative to said standard control indicates that said subject has or is at risk of developing an LPA-associated disease; and (iii) based at least in part on said expression level in step (ii), determining whether said subject has or is at risk for developing an LPA-associated disease.

### Embodiment 38

The method of embodiment 37, further comprising selecting a subject that has or is at risk for developing an LPA-associated disease.

### Embodiment 39

The method of embodiment 37, wherein said expression level of one or more LPA-associated disease marker RNAs set forth in Table 3 or Table 4 is detected from a biological sample of said subject.

### Embodiment 40

The method of embodiment 39, wherein said biological sample is a blood-derived biological sample, a urine-derived biological sample or a saliva-derived biological sample of said subject.

### Embodiment 41

The method of embodiment 40, wherein said blood-derived biological sample is whole blood, serum or plasma.

### Embodiment 42

The method of embodiment 37, further comprising administering to said subject an effective amount of a modulator of said LPA-associated disease marker RNA set forth in Table 3 or Table 4.

### Embodiment 43

The method of embodiment 42, wherein said modulator is an antagonist.

### Embodiment 44

The method of embodiment 43, wherein said antagonist is a peptide, small molecule, nucleic acid, antibody or aptamer.

### Embodiment 45

The method of embodiment 42, wherein said modulator is an agonist.

### Embodiment 46

The method of embodiment 45, wherein said agonist is a peptide, small molecule, nucleic acid, antibody or aptamer.

### Embodiment 47

The method of embodiment 42, further comprising administering to said subject an effective amount of a further therapeutic agent.

### Embodiment 48

A method of determining an LPA-associated disease activity in a patient, said method comprising: (i) determining a first expression level of a protein set forth in Table 1 or Table 2 in said patient at a first time point; (ii) determining a second expression level of a protein set forth in Table 1 or Table 2 in said patient at a second time point; (iii) comparing said second expression level of a protein set forth in Table 1 or Table 2 to said first expression level of a protein set forth in Table 1 or Table 2, thereby determining said LPA-associated disease activity in said patient.

### Embodiment 49

A method of determining an LPA-associated disease activity in a patient, said method comprising: (i) determining a first expression level of an RNA set forth in Table 3 or Table 4 in said patient at a first time point; (ii) determining a second expression level of an RNA set forth in Table 3 or Table 4 in said patient at a second time point; (iii) comparing said second expression level of an RNA set forth in Table 3 or Table 4 to said first expression level of an RNA set forth in Table 3 or Table 4, thereby determining said LPA-associated disease activity in said patient.

### Embodiment 50

A method of treating an LPA-associated disease in a subject in need thereof, said method comprising administering to said subject an effective amount of an modulator of an LPA-associated disease marker protein set forth in Table 1 or Table 2, thereby treating an LPA-associated disease in said subject.

### Embodiment 51

A method of treating an LPA-associated disease in a subject in need thereof, said method comprising administering to said subject an effective amount of an modulator of an LPA-associated disease marker RNA set forth in Table 3 or Table 4, thereby treating an LPA-associated disease in said subject.

### Embodiment 52

A method of treating an LPA-associated disease in a subject in need thereof, said method comprising: (i) determining whether a subject expresses an elevated level or a decreased level of an LPA-associated disease marker protein set forth in Table 1 or Table 2 relative to a standard control; and (ii) when an elevated expression level or a decreased expression level of said LPA-associated disease marker protein set forth in Table 1 or Table 2 is found relative to said standard control, administering to said subject an LPA-associated disease treatment, an antagonist or an agonist of an LPA-associated disease marker protein set forth in Table 1 or Table 2, thereby treating said subject.

### Embodiment 53

A method of treating an LPA-associated disease in a subject in need thereof, said method comprising: (i) determining whether a subject expresses an elevated level or a decreased level of an LPA-associated disease marker RNA set forth in Table 3 or Table 4 relative to a standard control; and (ii) when an elevated expression level or a decreased expression level of said LPA-associated disease marker RNA set forth in Table 3 or Table 4 is found relative to said standard control, administering to said subject an LPA-associated disease treatment, an antagonist or an agonist of an LPA-associated disease marker RNA set forth in Table 3 or Table 4, thereby treating said subject.

### Embodiment 54

A method of determining whether an LPA-associated disease patient is at risk for progression of the LPA-associated disease, the method comprising: (i) detecting an expression level of one or more LPA-associated disease marker proteins set forth in Table 1 or Table 2 in an LPA-associated disease patient; (ii) determining whether said expression level is modulated relative to a standard control, wherein a modulated expression level of an LPA-associated disease marker protein set forth in Table 1 or Table 2 relative to said standard control indicates that said LPA-associated disease patient is at risk for progression of said LPA-associated disease; and (iii) based at least in part on said expression level in step (ii), determining whether said LPA-associated disease patient is at risk for progression of said LPA-associated disease.

### Embodiment 55

A method of determining whether an LPA-associated disease patient is at risk for progression of the LPA-associated disease, the method comprising: (i) detecting an expression level of one or more LPA-associated disease marker RNAs set forth in Table 3 or Table 4 in an LPA-associated disease patient; (ii) determining whether said expression level is modulated relative to a standard control, wherein a modulated expression level of an LPA-associated disease marker RNA set forth in Table 3 or Table 4 relative to said standard control indicates that said LPA-associated disease patient is at risk for progression of said LPA-associated disease; and (iii) based at least in part on said expression level in step (ii), determining whether said LPA-associated disease patient is at risk for progression of said LPA-associated disease.

### Embodiments II

### Embodiment 1

A method of determining an expression level of an LPA-associated disease marker protein of SEQ ID NO:1-202 in a subject that has or is at risk for developing an LPA-associated disease, said method comprising: (i) obtaining a biological sample from said subject; and (ii) determining an expression level of an LPA-associated disease marker protein of SEQ ID NO:1-202 in said biological sample.

### Embodiment 2

The method of embodiment 1, wherein said determining comprises: (a) contacting said LPA-associated disease marker protein with a marker protein binding agent in said biological sample, thereby forming a disease marker protein-binding agent complex; and (b) detecting said disease marker protein-binding agent complex.

### Embodiment 3

The method of embodiment 2, wherein said marker protein binding agent comprises a detectable moiety.

### Embodiment 4

The method of embodiment 2, wherein said marker protein binding agent comprises a capturing moiety.

### Embodiment 5

The method of embodiment 4, wherein said capturing moiety is a cleavable capturing moiety.

### Embodiment 6

The method of one of embodiments 2-5, wherein said detecting comprises contacting said disease marker protein-binding agent complex with a capturing agent, thereby forming a captured disease marker protein-binding agent complex.

### Embodiment 7

The method of embodiment 6, wherein said detecting further comprises: (1) contacting said captured disease marker protein-binding agent complex with a tagging moiety; thereby forming a tagged disease marker protein-binding agent complex; and (2) separating said tagged disease marker protein-binding agent complex from said biological sample.

### Embodiment 8

The method of embodiment 7, wherein said detecting further comprises after said separating of step (2) separating said capturing binding agent from said tagged disease marker protein-binding agent complex, thereby forming a cleaved disease marker protein-binding agent complex.

### Embodiment 9

The method of embodiment 8, wherein said detecting further comprises: (3) separating said marker protein binding agent from said cleaved disease marker protein-binding agent complex; thereby forming a released marker protein binding agent; and (4) determining an amount of said released marker protein binding agent.

### Embodiment 10

The method of one of embodiments 1-9, further comprising selecting a subject that has or is at risk for developing an LPA-associated disease.

### Embodiment 11

The method of one of embodiments 1-10, wherein said biological sample is a blood-derived biological sample, a urine-derived biological sample or a saliva-derived biological sample of said subject.

### Embodiment 12

The method of embodiment 11, wherein said blood-derived biological sample is whole blood, serum or plasma.

### Embodiment 13

The method of one of embodiments 1-12, further comprising administering to said subject an effective amount of a modulator of said LPA-associated disease marker protein of SEQ ID NO:1-202.

### Embodiment 14

The method of embodiment 13, wherein said modulator is an antagonist.

### Embodiment 15

The method of embodiment 14 wherein said antagonist is a peptide, small molecule, nucleic acid, antibody or aptamer

### Embodiment 16

The method of embodiment 14, wherein said expression level of said LPA-associated disease marker protein of SEQ ID NO:1-202 is elevated relative to a standard control.

### Embodiment 17

The method of embodiment 13, wherein said modulator is an agonist.

### Embodiment 18

The method of embodiment 17, wherein said agonist is a peptide, small molecule, nucleic acid, antibody or aptamer.

### Embodiment 19

The method of embodiment 17, wherein said expression level of said LPA-associated disease marker protein of SEQ ID NO:1-202 is decreased relative to a standard control.

### Embodiment 20

The method of one of embodiments 13-19, further comprising administering to said subject an effective amount of a further therapeutic agent.

### Embodiment 21

The method of one of embodiments 1-20, wherein said LPA-associated disease marker protein is SEQ ID NO:9, SEQ ID NO:23, SEQ ID NO:32, SEQ ID NO:47, SEQ ID NO:58, SEQ ID NO:90, SEQ ID NO:93, SEQ ID NO:102, SEQ ID NO:118, SEQ ID NO:131, SEQ ID NO:136, SEQ ID NO:139, SEQ ID NO:141, SEQ ID NO:143, SEQ ID NO:149, SEQ ID NO:152, SEQ ID NO:160, SEQ ID NO:164, SEQ ID NO:183 or SEQ ID NO:185.

### Embodiment 22

A method of determining an expression level of an LPA-associated disease marker RNA of SEQ ID NO:203-499 in a subject that has or is at risk for developing an LPA-associated disease, said method comprising: (i) obtaining a biological sample from said subject; and (ii) determining an expression level of an LPA-associated disease marker RNA of SEQ ID NO:203-499 in said biological sample.

### Embodiment 23

The method of embodiment 22, wherein said determining comprises: (a) contacting said LPA-associated disease marker RNA with a marker RNA binding agent in said biological sample, thereby forming a disease marker RNA-binding agent complex; and (b) detecting said disease marker RNA-binding agent complex.

### Embodiment 24

The method of embodiment 23, wherein said marker RNA binding agent comprises a detectable moiety.

### Embodiment 25

The method of embodiment 23, wherein said marker RNA binding agent comprises a capturing moiety.

### Embodiment 26

The method of embodiment 25, wherein said capturing moiety is a cleavable capturing moiety.

### Embodiment 27

The method of one of embodiments 23-26, wherein said detecting comprises contacting said disease marker RNA-binding agent complex with a capturing agent, thereby forming a captured disease marker RNA-binding agent complex.

### Embodiment 28

The method of embodiment 27, wherein said detecting further comprises: (1) contacting said captured disease marker RNA-binding agent complex with a tagging moiety; thereby forming a tagged disease marker RNA-binding agent complex; and (2) separating said tagged disease marker RNA-binding agent complex from said biological sample.

### Embodiment 29

The method of embodiment 28, wherein said detecting further comprises after said separating of step (2) separating said capturing binding agent from said tagged disease marker RNA-binding agent complex, thereby forming a cleaved disease marker RNA-binding agent complex.

### Embodiment 30

The method of embodiment 29, wherein said detecting further comprises: (3) separating said marker RNA binding agent from said cleaved disease marker RNA-binding agent complex; thereby forming a released marker RNA binding agent; and (4) determining an amount of said released marker RNA binding agent.

### Embodiment 31

The method of one of embodiments 22-30, further comprising selecting a subject that has or is at risk for developing an LPA-associated disease.

### Embodiment 32

The method of one of embodiments 22-31, wherein said biological sample is a blood-derived biological sample, a urine-derived biological sample or a saliva-derived biological sample of said subject

### Embodiment 33

The method of embodiment 32, wherein said blood-derived biological sample is whole blood, serum or plasma.

### Embodiment 34

The method of one of embodiments 22-33, further comprising administering to said subject an effective amount of a modulator of said LPA-associated disease marker RNA of SEQ ID NO:203-499.

### Embodiment 35

The method of embodiment 34, wherein said modulator is an antagonist.

### Embodiment 36

The method of embodiment 35, wherein said antagonist is a peptide, small molecule, nucleic acid, antibody or aptamer.

### Embodiment 37

The method of embodiment 35, wherein said expression level of said LPA-associated disease marker RNA of SEQ ID NO:203-499 is elevated relative to a standard control.

### Embodiment 38

The method of embodiment 34, wherein said modulator is an agonist.

### Embodiment 39

The method of embodiment 38, wherein said agonist is a peptide, small molecule, nucleic acid, antibody or aptamer.

### Embodiment 40

The method of embodiment 38, wherein said expression level of said LPA-associated disease marker RNA of SEQ ID NO:203-499 is decreased relative to a standard control.

### Embodiment 41

The method of one of embodiments 34-40, further comprising administering to said subject an effective amount of a further therapeutic agent.

### Embodiment 42

The method of one of embodiments 22-41, wherein said LPA-associated disease marker RNA is SEQ ID NO:209, SEQ ID NO:214, SEQ ID NO:216, SEQ ID NO:217, SEQ ID NO:218, SEQ ID NO:219, SEQ ID NO:222, SEQ ID NO:236, SEQ ID NO:237, SEQ ID NO:242, SEQ ID NO:243, SEQ ID NO:267, SEQ ID NO:269, SEQ ID NO:270, SEQ ID NO:271, SEQ ID NO:303, SEQ ID NO:309, SEQ ID NO:353, SEQ ID NO:364, SEQ ID NO:378, SEQ ID NO:396, SEQ ID NO:417, SEQ ID NO:432 or SEQ ID NO:448.

### Embodiment 43

A method of determining whether a subject has or is at risk of developing an LPA-associated disease, said method comprising: (i) detecting an expression level of one or more LPA-associated disease marker proteins of SEQ ID NO:1-202 in a subject; (ii) determining whether said expression level is increased or decreased relative to a standard control, wherein an elevated expression level of an LPA-associated disease marker protein of SEQ ID NO:1-202 or a decreased expression level of an LPA-associated disease marker protein of SEQ ID NO:1-202 relative to said standard control indicates that said subject has or is at risk of developing an LPA-associated disease; and (iii) based at least in part on said expression level in step (ii), determining whether said subject has or is at risk for developing an LPA-associated disease.

### Embodiment 44

The method of embodiment 43, further comprising selecting a subject that has or is at risk for developing an LPA-associated disease.

### Embodiment 45

The method of embodiment 43, wherein said expression level of one or more LPA-associated disease marker proteins of SEQ ID NO:1-202 is detected from a biological sample of said subject.

### Embodiment 46

The method of embodiment 45, wherein said biological sample is a blood-derived biological sample, a urine-derived biological sample or a saliva-derived biological sample of said subject.

### Embodiment 47

The method of embodiment 46, wherein said blood-derived biological sample is whole blood, serum or plasma.

### Embodiment 48

The method of embodiment 43, further comprising administering to said subject an effective amount of a modulator of said LPA-associated disease marker protein of SEQ ID NO:1-202.

### Embodiment 49

The method of embodiment 48, wherein said modulator is an antagonist.

### Embodiment 50

The method of embodiment 49, wherein said antagonist is a peptide, small molecule, nucleic acid, antibody or aptamer.

### Embodiment 51

The method of embodiment 48, wherein said modulator is an agonist.

### Embodiment 52

The method of embodiment 51, wherein said agonist is a peptide, small molecule, nucleic acid, antibody or aptamer.

### Embodiment 53

The method of embodiment 48, further comprising administering to said subject an effective amount of a further therapeutic agent.

### Embodiment 54

A method of determining whether a subject has or is at risk of developing an LPA-associated disease, said method comprising: (i) detecting an expression level of one or more LPA-associated disease marker RNAs of SEQ ID NO:203-499 in a subject; (ii) determining whether said expression level is increased or decreased relative to a standard control, wherein an elevated expression level of an LPA-associated disease marker RNA of SEQ ID NO:203-499 or a decreased expression level of an LPA-associated disease marker RNA of SEQ ID NO:203-499 relative to said standard control indicates that said subject has or is at risk of developing an LPA-associated disease; and (iii) based at least in part on said expression level in step (ii), determining whether said subject has or is at risk for developing an LPA-associated disease.

### Embodiment 55

The method of embodiment 54, further comprising selecting a subject that has or is at risk for developing an LPA-associated disease.

### Embodiment 56

The method of embodiment 54, wherein said expression level of one or more LPA-associated disease marker RNAs of SEQ ID NO:203-499 is detected from a biological sample of said subject.

### Embodiment 57

The method of embodiment 56, wherein said biological sample is a blood-derived biological sample, a urine-derived biological sample or a saliva-derived biological sample of said subject.

### Embodiment 58

The method of embodiment 57, wherein said blood-derived biological sample is whole blood, serum or plasma.

### Embodiment 59

The method of embodiment 54, further comprising administering to said subject an effective amount of a modulator of said LPA-associated disease marker RNA of SEQ ID NO:203-499.

### Embodiment 60

The method of embodiment 59, wherein said modulator is an antagonist.

### Embodiment 61

The method of embodiment 60, wherein said antagonist is a peptide, small molecule, nucleic acid, antibody or aptamer.

### Embodiment 62

The method of embodiment 59, wherein said modulator is an agonist.

### Embodiment 63

The method of embodiment 62, wherein said agonist is a peptide, small molecule, nucleic acid, antibody or aptamer.

### Embodiment 64

The method of embodiment 59, further comprising administering to said subject an effective amount of a further therapeutic agent.

### Embodiment 65

A method of determining an LPA-associated disease activity in a patient, said method comprising: (i) determining a first expression level of a protein of SEQ ID NO:1-202 in said patient at a first time point; (ii) determining a second expression level of a protein of SEQ ID NO:1-202 in said patient at a second time point; (iii) comparing said second expression level of a protein of SEQ ID NO:1-202 to said first expression level of a protein of SEQ ID NO:1-202, thereby determining said LPA-associated disease activity in said patient.

### Embodiment 66

A method of determining an LPA-associated disease activity in a patient, said method comprising: (i) determining a first expression level of an RNA of SEQ ID NO:203-499 in said patient at a first time point; (ii) determining a second expression level of an RNA of SEQ ID NO:203-499 in said patient at a second time point; (iii) comparing said second expression level of an RNA of SEQ ID NO:203-499 to said first expression level of an RNA of SEQ ID NO:203-499, thereby determining said LPA-associated disease activity in said patient.

### Embodiment 67

A method of treating an LPA-associated disease in a subject in need thereof, said method comprising administering to said subject an effective amount of an modulator of an LPA-associated disease marker protein of SEQ ID NO:1-202, thereby treating an LPA-associated disease in said subject.

### Embodiment 68

A method of treating an LPA-associated disease in a subject in need thereof, said method comprising administering to said subject an effective amount of an modulator of an LPA-associated disease marker RNA of SEQ ID NO:203-499, thereby treating an LPA-associated disease in said subject.

### Embodiment 69

A method of treating an LPA-associated disease in a subject in need thereof, said method comprising: (i) determining whether a subject expresses an elevated level or a decreased level of an LPA-associated disease marker protein of SEQ ID NO:1-202 relative to a standard control; and (ii) when an elevated expression level or a decreased expression level of said LPA-associated disease marker protein of SEQ ID NO:1-202 is found relative to said standard control, administering to said subject an LPA-associated disease treatment, an antagonist or an agonist of an LPA-associated disease marker protein of SEQ ID NO:1-202, thereby treating said subject.

### Embodiment 70

A method of treating an LPA-associated disease in a subject in need thereof, said method comprising: (i) determining whether a subject expresses an elevated level or a decreased level of an LPA-associated disease marker RNA of SEQ ID NO:203-499 relative to a standard control; and (ii) when an elevated expression level or a decreased expression level of said LPA-associated disease marker RNA of SEQ ID NO:203-499 is found relative to said standard control, administering to said subject an LPA-associated disease treatment, an antagonist or an agonist of an LPA-associated disease marker RNA of SEQ ID NO:203-499, thereby treating said subject.

### Embodiment 71

A method of determining whether an LPA-associated disease patient is at risk for progression of the LPA-associated disease, the method comprising: (i) detecting an expression level of one or more LPA-associated disease marker proteins set forth of SEQ ID NO:1-202 in an LPA-associated disease patient; (ii) determining whether said expression level is modulated relative to a standard control, wherein a modulated expression level of an LPA-associated disease marker protein of SEQ ID NO:1-202 relative to said standard control indicates that said LPA-associated disease patient is at risk for progression of said LPA-associated disease; and (iii) based at least in part on said expression level in step (ii), determining whether said LPA-associated disease patient is at risk for progression of said LPA-associated disease.

### Embodiment 72

A method of determining whether an LPA-associated disease patient is at risk for progression of the LPA-associated disease, the method comprising: (i) detecting an expression level of one or more LPA-associated disease marker RNAs of SEQ ID NO:203-499 in an LPA-associated disease patient; (ii) determining whether said expression level is modulated relative to a standard control, wherein a modulated expression level of an LPA-associated disease marker RNA of SEQ ID NO:203-499 relative to said standard control indicates that said LPA-associated disease patient is at risk for progression of said LPA-associated disease; and (iii) based at least in part on said expression level in step (ii), determining whether said LPA-associated disease patient is at risk for progression of said LPA-associated disease.

### Embodiment 73

A complex in vitro comprising a marker protein binding agent bound to a LPA-associated disease marker protein of SEQ ID NO:1-202 or fragment thereof, wherein said LPA-associated disease marker protein is extracted from a human subject having or at risk of developing an LPA-associated disease.

### Embodiment 74

The complex of embodiment 73, wherein said subject has an LPA-associated disease.

### Embodiment 75

A complex in vitro comprising a marker RNA binding agent bound to a LPA-associated disease marker RNA of SEQ ID NO:203-499 or fragment thereof, wherein said LPA-associated disease marker RNA is extracted from a human subject having or at risk of developing an LPA-associated disease.

### Embodiment 76

The complex of embodiment 75, wherein said subject has an LPA-associated disease.

### Embodiment 77

A kit comprising: (a) a marker protein binding agent capable of binding to a substance within a biological sample from a human subject having or at risk of developing an LPA-associated disease; wherein said substance is an LPA-associated disease marker protein of SEQ ID NO:1-202 or fragment thereof; (b) a detecting reagent or a detecting apparatus capable of indicating binding of said marker protein binding agent to said substance.

### Embodiment 78

A kit comprising: (a) a marker RNA binding agent capable of binding to a substance within a biological sample from a human subject having or at risk of developing an LPA-associated disease; wherein said substance is an LPA-associated disease marker RNA of SEQ ID NO:203-499 or fragment thereof; (b) a detecting reagent or a detecting apparatus capable of indicating binding of said marker RNA binding agent to said substance.

1. A method of determining an expression level of an LPA-associated disease marker protein of SEQ ID NO:1-202 in a subject that has or is at risk for developing an LPA-associated disease, said method comprising:
(i) obtaining a biological sample from said subject; and
(ii) determining an expression level of an LPA-associated disease marker protein of SEQ ID NO:1-202 in said biological sample.

2. The method of claim 1, wherein said determining comprises: (a) contacting said LPA-associated disease marker protein with a marker protein binding agent in said biological sample, thereby forming a disease marker protein-binding agent complex; and (b) detecting said disease marker protein-binding agent complex.

3-5. (canceled)

6. The method of claim 2, wherein said detecting comprises contacting said disease marker protein-binding agent complex with a capturing agent, thereby forming a captured disease marker protein-binding agent complex.

7. The method of claim 6, wherein said detecting further comprises: (1) contacting said captured disease marker protein-binding agent complex with a tagging moiety; thereby forming a tagged disease marker protein-binding agent complex; and (2) separating said tagged disease marker protein-binding agent complex from said biological sample.

8. The method of claim 7, wherein said detecting further comprises after said separating of step (2) separating said capturing binding agent from said tagged disease marker protein-binding agent complex, thereby forming a cleaved disease marker protein-binding agent complex.

9. The method of claim 8, wherein said detecting further comprises: (3) separating said marker protein binding agent from said cleaved disease marker protein-binding agent complex; thereby forming a released marker protein binding agent; and (4) determining an amount of said released marker protein binding agent.

10. The method of claim 1, further comprising selecting a subject that has or is at risk for developing an LPA-associated disease.

11-12. (canceled)

13. The method of claim 10, further comprising administering to said subject an effective amount of a modulator of said LPA-associated disease marker protein of SEQ ID NO:1-202.

14-20. (canceled)

21. The method of claim 1, wherein said LPA-associated disease marker protein is SEQ ID NO:9, SEQ ID NO:23, SEQ ID NO:32, SEQ ID NO:47, SEQ ID NO:58, SEQ ID NO:90, SEQ ID NO:93, SEQ ID NO:102, SEQ ID NO:118, SEQ ID NO:131, SEQ ID NO:136, SEQ ID NO:139, SEQ ID NO:141, SEQ ID NO:143, SEQ ID NO:149, SEQ ID NO:152, SEQ ID NO:160, SEQ ID NO:164, SEQ ID NO:183 or SEQ ID NO:185.

22. A method of determining an expression level of an LPA-associated disease marker RNA of SEQ ID NO:203-499 in a subject that has or is at risk for developing an LPA-associated disease, said method comprising:
(i) obtaining a biological sample from said subject; and
(ii) determining an expression level of an LPA-associated disease marker RNA of SEQ ID NO:203-499 in said biological sample.

23. The method of claim 22, wherein said determining comprises: (a) contacting said LPA-associated disease marker RNA with a marker RNA binding agent in said biological sample, thereby forming a disease marker RNA-binding agent complex; and (b) detecting said disease marker RNA-binding agent complex.

24-26. (canceled)

27. The method of claim 23, wherein said detecting comprises contacting said disease marker RNA-binding agent complex with a capturing agent, thereby forming a captured disease marker RNA-binding agent complex.

28. The method of claim 27, wherein said detecting further comprises: (1) contacting said captured disease marker RNA-binding agent complex with a tagging moiety; thereby forming a tagged disease marker RNA-binding agent complex; and (2) separating said tagged disease marker RNA-binding agent complex from said biological sample.

29. The method of claim 28, wherein said detecting further comprises after said separating of step (2) separating said capturing binding agent from said tagged disease marker RNA-binding agent complex, thereby forming a cleaved disease marker RNA-binding agent complex.

30. The method of claim 29, wherein said detecting further comprises: (3) separating said marker RNA binding agent from said cleaved disease marker RNA-binding agent complex; thereby forming a released marker RNA binding agent; and (4) determining an amount of said released marker RNA binding agent.

31. The method of claim 22, further comprising selecting a subject that has or is at risk for developing an LPA-associated disease.

32-33. (canceled)

34. The method of claim 31, further comprising administering to said subject an effective amount of a modulator of said LPA-associated disease marker RNA of SEQ ID NO:203-499.

35-41. (canceled)

42. The method of claim 22, wherein said LPA-associated disease marker RNA is SEQ ID NO:209, SEQ ID NO:214, SEQ ID NO:216, SEQ ID NO:217, SEQ ID NO:218, SEQ ID NO:219, SEQ ID NO:222, SEQ ID NO:236, SEQ ID NO:237, SEQ ID NO:242, SEQ ID NO:243, SEQ ID NO:267, SEQ ID NO:269, SEQ ID NO:270, SEQ ID NO:271, SEQ ID NO:303, SEQ ID NO:309, SEQ ID NO:353, SEQ ID NO:364, SEQ ID NO:378, SEQ ID NO:396, SEQ ID NO:417, SEQ ID NO:432 or SEQ ID NO:448.

43. A method of determining whether a subject has or is at risk of developing an LPA-associated disease, said method comprising:
(i) detecting an expression level of one or more LPA-associated disease marker proteins of SEQ ID NO:1-202 in a subject;
(ii) determining whether said expression level is increased or decreased relative to a standard control, wherein an elevated expression level of an LPA-associated disease marker protein of SEQ ID NO:1-202 or a decreased expression level of an LPA-associated disease marker protein of SEQ ID NO:1-202 relative to said standard control indicates that said subject has or is at risk of developing an LPA-associated disease; and
(iii) based at least in part on said expression level in step (ii), determining whether said subject has or is at risk for developing an LPA-associated disease.

44-47. (canceled)

48. The method of claim 43, further comprising administering to said subject an effective amount of a modulator of said LPA-associated disease marker protein of SEQ ID NO:1-202.

49-53. (canceled)

54. A method of determining whether a subject has or is at risk of developing an LPA-associated disease, said method comprising:
(i) detecting an expression level of one or more LPA-associated disease marker RNAs of SEQ ID NO:203-499 in a subject;
(ii) determining whether said expression level is increased or decreased relative to a standard control, wherein an elevated expression level of an LPA-associated disease marker RNA of SEQ ID NO:203-499 or a decreased expression level of an LPA-associated disease marker RNA of SEQ ID NO:203-499 relative to said standard control indicates that said subject has or is at risk of developing an LPA-associated disease; and
(iii) based at least in part on said expression level in step (ii), determining whether said subject has or is at risk for developing an LPA-associated disease.

55-58. (canceled)

59. The method of claim 54, further comprising administering to said subject an effective amount of a modulator of said LPA-associated disease marker RNA of SEQ ID NO:203-499.

60-66. (canceled)

67. A method of treating an LPA-associated disease in a subject in need thereof, said method comprising administering to said subject an effective amount of an modulator of an LPA-associated disease marker protein of SEQ ID NO:1-202, thereby treating an LPA-associated disease in said subject.

68. A method of treating an LPA-associated disease in a subject in need thereof, said method comprising administering to said subject an effective amount of an modulator of an LPA-associated disease marker RNA of SEQ ID NO:203-499, thereby treating an LPA-associated disease in said subject.

69-70. (canceled)

71. A method of determining whether an LPA-associated disease patient is at risk for progression of the LPA-associated disease, the method comprising:
(i) detecting an expression level of one or more LPA-associated disease marker proteins set forth of SEQ ID NO:1-202 in an LPA-associated disease patient;
(ii) determining whether said expression level is modulated relative to a standard control,
wherein a modulated expression level of an LPA-associated disease marker protein of SEQ ID NO:1-202 relative to said standard control indicates that said LPA-associated disease patient is at risk for progression of said LPA-associated disease; and (iii) based at least in part on said expression level in step (ii), determining whether said LPA-associated disease patient is at risk for progression of said LPA-associated disease.

72. A method of determining whether an LPA-associated disease patient is at risk for progression of the LPA-associated disease, the method comprising:
(i) detecting an expression level of one or more LPA-associated disease marker RNAs of SEQ ID NO:203-499 in an LPA-associated disease patient;
(ii) determining whether said expression level is modulated relative to a standard control,
wherein a modulated expression level of an LPA-associated disease marker RNA of SEQ ID NO:203-499 relative to said standard control indicates that said LPA-associated disease patient is at risk for progression of said LPA-associated disease; and (iii) based at least in part on said expression level in step (ii), determining whether said LPA-associated disease patient is at risk for progression of said LPA-associated disease.

73-76. (canceled)

77. A kit comprising: (a) a marker protein binding agent capable of binding to a substance within a biological sample from a human subject having or at risk of developing an LPA-associated disease; wherein said substance is an LPA-associated disease marker protein of SEQ ID NO:1-202 or fragment thereof; (b) a detecting reagent or a detecting apparatus capable of indicating binding of said marker protein binding agent to said substance.

78. A kit comprising: (a) a marker RNA binding agent capable of binding to a substance within a biological sample from a human subject having or at risk of developing an LPA-associated disease; wherein said substance is an LPA-associated disease marker RNA of SEQ ID NO:203-499 or fragment thereof; (b) a detecting reagent or a detecting apparatus capable of indicating binding of said marker RNA binding agent to said substance.

79. The method of one of claim 1, 22, 43, 54, 67, 68, 71 or 72, wherein said LPA-associated disease is a fibrotic pulmonary disease.

80. The method of claim 79, wherein said fibrotic pulmonary disease is idiopathic pulmonary fibrosis or familial interstitial pneumonia.

81. The method of claim 79, wherein said fibrotic pulmonary disease is a progressive form of idiopathic pulmonary fibrosis.

82. The kit of one of claim 77 or 78, wherein said LPA-associated disease is a fibrotic pulmonary disease.

83. The kit of claim 82, wherein said fibrotic pulmonary disease is idiopathic pulmonary fibrosis or familial interstitial pneumonia.

84. The kit of claim 82, wherein said fibrotic pulmonary disease is a progressive form of idiopathic pulmonary fibrosis.
